(19) United States
(12) Patent Application Publication
(10) Pub. No.: US 2025/0361480 A1
(43) Pub. Date: Nov. 27, 2025

(54) REINFORCED ENGINEERED CELLULARIZED-TISSUE

(71) Applicant: Ramot at Tel-Aviv University Ltd., Tel-Aviv (IL)

(72) Inventors: Tal DVIR, Tel-Aviv (IL); Eric SILBERMAN, Tel-Aviv (IL)

(73) Assignee: Ramot at Tel-Aviv University Ltd., Tel-Aviv (IL)

(21) Appl. No.: 19/294,358

(22) Filed: Aug. 8, 2025

Related U.S. Application Data
(63) Continuation of application No. PCT/IL2024/050153, filed on Feb. 8, 2024.
(60) Provisional application No. 63/444,826, filed on Feb. 10, 2023.

(51) Int. Cl. C12N 5/00; A61K 35/12; C12N 5/071

(52) U.S. Cl. CPC C12N 5/0062; A61K 35/12; C12N 5/0697; C12N 2501/90; C12N 2513/00; C12N 2537/10

(57) ABSTRACT

A method for generating reinforced engineered cellularized construct, which utilizes a biocompatible small-molecule reinforcing agent that do not affect the viability of the cells, a reinforced engineered cellularized construct obtained thereby and used the engineered cellularized construct are provided.

### RELATED APPLICATIONS

This application is a Continuation of PCT Patent Application No. PCT/IL2024/050153 having International filing date of Feb. 8, 2024 which claims the benefit of priority under 35 USC § 119(e) of U.S. Provisional Patent Application No. 63/444,826 filed Feb. 10, 2023. The contents of the applications are all incorporated by reference as if fully set forth herein in their entirety.

### FIELD AND BACKGROUND OF THE INVENTION

The present invention, in some embodiments thereof, relates to 3D bioprinting and, more particularly, but not exclusively, to formulations usable in 3D bioprinting of cellularized objects, to 3D bioprinting methods employing same, and to cellularized objects obtained thereby and uses thereof.

Tissue-engineered cardiac patches are envisioned to be a promising treatment option for patients who have suffered a myocardial infarction. These patches synergistically combine mechanical support and biological functionality to repair a damaged myocardium [Li et al, VIEW 2022, 3, 20200153]. Ideally, cardiac patches should approximate the native human myocardium, a highly-vascularized, densely cell-laden tissue, which reaches a thickness of 1 cm. To recapitulate this structure, advanced fabrication techniques, such as 3D bioprinting, are used.

Three-dimensional (3D) printing is an additive manufacturing technology that allows bottom-up construction of complex structures. The boundaries of the printed model are defined by a computer-aided design (CAD) software and accordingly the printer deposits a building material in a layer-by-layer manner. Three-dimensional (3D) bioprinting uses biological materials, optionally in combination with chemicals and/or cells, that are printed layer-by-layer with a precise positioning and a tight control of functional components placement to create a 3D biological structure.

Three dimensional (3D) bioprinting is gaining momentum in many medicinal applications, especially in regenerative medicine, to address the need for complex scaffolds, tissues and organs suitable for transplantation.

Inherent to 3D printing in general is that the mechanical properties of the printing media (the building material, bioink) are very different from the post-printed cured (hardened) material.

Different technologies have been developed for 3D bioprinting, including 3D Inkjet printing, Extrusion printing, Laser-assisted printing, digital light processing, and Projection stereolithography [see, for example, Murphy et al., Nature Biotechnology. 2014 32(8); Miller et al. ACS Biomater. Sci. Eng. 2016, 2, 1658-1661]. Each technology has its different requirements for the printing media, which is derived from the specific application mechanism and the curing/gelation process required to maintain the 3D structure of the scaffold post printing.

Recent advances in the field have enabled utilization of various printing technologies for delivering living cells with materials. One of the promising technologies to print tissues is by extrusion. Compared to inkjet and laser-assisted printing, which deposit dissociated liquid droplets, extrusion printers use robotically controlled extrusion heads to deposit continues strands of materials in which cells can be incorporated.

Bioprinting, such as extrusion-based bioprinting, enables the generation of carefully controlled, heterogeneous structures in accordance with a digital design [Shapira and Dvir, Adv. Sci. 2021, 8, 2003751]. Extrusion-based bioprinting technology has been used to fabricate cardiac patches while incorporating a vascular network ab initio, which is required for maintaining cell viability when dealing with tissues thicker than about 400 microns [He and Chen, Adv. Healthcare Mater. 2020, 9, 2001175; Williams et al., Tissue Eng., Part B 2022, 28, 336]. However, because extrusion-based bioprinting relies on forcing materials through a print head nozzle, it can only be used with flowable materials, which must be optimized post-printing to achieve their desired strength.

Extracellular matrix (ECM)-based hydrogels are often used as a scaffold material in tissue engineering due to their wealth of biologically relevant molecules that help cells adhere to and mature within the scaffold [Hussey et al., Nat. Rev. Mater. 2018, 3, 159; Crapo et al., Biomaterials 2011, 32, 3233]. However, as these hydrogels tend to have weak mechanical properties, a variety of different techniques have been developed to make ECM-based hydrogels more robust [Walimbe and Panitch, Bioengineering 2020, 7, 156; Kreger et al., Biopolymers 2010, 93, 690].

Often, in order to preserve cell viability, a structure's mechanical properties are optimized in the absence of cells, which are then seeded at a later stage. This approach, however, has two distinct drawbacks. First, cells will not migrate evenly into the core of a thick structure, and second, there is no way to precisely localize specific cell types when allowing the cells to migrate freely into the structure.

Other formulations allow for cells to be encapsulated within the ECM-based bioink before printing. For instance, methacrylated polymers, typified by GelMA, are often cross-linked in the presence of cells by using a short exposure to UV radiation [Bertassoni et al., Biofabrication 2014, 6, 024105; Schuurman et al., Macromol. Biosci. 2013, 13, 551].

However, the exposure of cells to UV and the subsequent generation of free radicals have the potential to harm the cells, limiting the utility of these techniques [Van Belleghem et al., Adv. Funct. Mater. 2020, 30, 1907145]. Additionally, because UV light can only minimally penetrate tissue, the cross-linking cannot be uniformly performed on a thick tissue [Rapp and DeForest, Adv. Healthcare Mater. 2020, 9, 1901553; Remmers and Neumann, Biomater. Sci. 2023, 11, 1607]. As a result, the cross-linking takes place during tissue assembly, and the process cannot be used as to modify fully assembled, functional tissues.

Another common alternative is using genipin, a naturally occurring compound from the Gardenia fruit. However, genipin's reactivity is difficult to control, and it spontaneously reacts with the amines present in almost all proteins. As a result, genipin cannot be effectively used in combination with cell media that contains serum [Wang et al., J. Biomed. Mater. Res., Part B 2011, 97B, 58; Sung et al., J. Biomed. Mater. Res. 1999, 46, 520; Birman et al., Adv. Funct. Mater. 2021, 31, 2100628].

Thus, printing complex tissues such as the myocardium, which consists of various cell types (e.g., cardiac fibroblasts and myocytes) together with a dense vasculature, remains a challenge, mainly due to the relatively inferior physical properties of biomaterials such as natural, ECM-derived substances that are being used as biocompatible and biodegradable “bio-inks” (printing media) for the printing process. Some of the present inventors have previously demonstrated a method for printing thick, vascularized cardiac tissues [Noor et al., Adv. Sci. 2019, 6, 1900344]. By incorporating cells in the bioink (printing media; building material) before printing, these tissues could be uniformly and fully cellularized. By utilizing an extrusion-based bioprinting methodology, it was shown that multiple cell types can be positioned in their appropriate locations, creating distinct tissue units. However, the tissue's mechanical properties were determined only by the physical gelation of the ECM-based material, and as such were liable to disintegrate when subjected to shear or compression forces, such as those exerted during the transplantation process, when suturing the tissues, or when implanting the engineered tissues via a minimally invasive procedure [Shevach et al., Biomed. Mater. 2015, 10, 034106; Edri et al., Adv. Mater. 2019, 31, 1970007].

Oxidized sucrose, which is also referred to in the art as SOx, is a polyaldehyde that reacts with amine moieties present in the native ECM via a Schiff base “click” reaction [Nezhad-Mokhtari et al. Eur. Polym. J. 2019, 117, 64; see, Background Art FIG. 2B].

To date, all studies that were conducted with SOx have applied the molecule to non-cellularized scaffolds to which cells were later added.

International Patent Application Publication No. WO 2009/085547 teaches the generation of decellularized omentum scaffolds for tissue engineering. International Patent Application No. WO 2009/085547 does not teach use of the decellularized omentum scaffolds for cardiac engineering.

International Patent Application Publication No. WO 2014/207744 teaches the generation of decellularized omentum scaffolds for tissue engineering. International Patent Application No. WO 2014/207744 does not teach conditions for decellularizing human omentum.

U.S. Patent Publication No. 20050013870 teaches a scaffold comprising decellularized extracellular matrix of a number of body tissues including omentum. The body tissues have been conditioned to produce a biological material such as a growth factor.

Porzionato et al. (Italian Journal of Anatomy and Embryology, Volume 116, 2011 and Eur J Histochem. 2013 Jan. 24; 57(1):e4. doi: 10.4081/ejh.2013.e4) teaches decellularized omentum.

Additional background art includes Gilbert et al., Biomaterials 27 (2006) 3675-3683 and Flynn et al., Biomaterials 31 (2010), 4715-4724.

U.S. Patent Publication No. 2009/0163990 and 2020/0101198-A1 teaches methods of decellularizing omentum.

Soluble forms of decellularized extracellular matrix are known in the art as described in Acta Biomaterialia, Volume 9, Issue 8, August 2013, Pages 7865-7873 and Singelyn et al., J Am Coll Cardiol. Feb. 21, 2012; 59(8): 751-763.

Additional background art includes Silberman et al., Adv. Mater. 2023, 35, 2302229 WO 2015/017421, EP Patent No. 1517778; DE 102012100859; WO 2019/234738.

### SUMMARY OF THE INVENTION

According to an aspect of some embodiments of the present invention there is provided a method for reinforcing an engineered cellularized construct fabricated from extracellular matrix (ECM) hydrogel and cells, the method comprising contacting the engineered cellularized construct with a biocompatible small-molecule reinforcing agent that is capable of chemically interacting with the ECM-based hydrogel under conditions that maintain viability of the cells, to thereby increase a compressive modulus of the ECM-based hydrogel by at least 10%, wherein the construct is devoid of retinal pigment epithelial (RPE) cells.

According to an aspect of some embodiments of the present invention there is provided a method of preparing a cellularized engineered construct, the method comprising:
encapsulating cells in the presence of an ECM-based hydrogel, to thereby provide a bioink composition;
depositing the bioink composition in a configured pattern corresponding to the shape of the engineered construct;
culturing the cells of the engineered construct; and
subsequent to the culturing, contacting the cellularized engineered construct with a reinforcing agent, the reinforcing agent being a biocompatible small-molecule reinforcing agent that is capable of chemically interacting with the ECM-based hydrogel under conditions that maintain viability of the cells, to thereby increase a compressive modulus of the ECM-based hydrogel by at least 10%, wherein the construct is devoid of RPE cells.

According to some embodiments of any of the embodiments described herein, the chemically interacting effects cross-linking of the ECM-based hydrogel.

According to some embodiments of any of the embodiments described herein, the reinforcing agent is capable of chemically interacting with the ECM-based hydrogel via a Click reaction.

According to some embodiments of any of the embodiments described herein, the Click reaction forms a Schiff base (an imine bond).

According to some embodiments of any of the embodiments described herein, the reinforcing agent is a polyaldehyde.

According to some embodiments of any of the embodiments described herein, the reinforcing agent is an oxidized, poly-aldehyde saccharide.

According to some embodiments of any of the embodiments described herein, the contacting is with a culturing medium that comprises the reinforcing agent.

According to some embodiments of any of the embodiments described herein, the reinforcing agent is an oxidized, poly-aldehyde saccharide and wherein a concentration of the reinforcing agent in the medium is less than 0.1% by weight of the total weight of the medium.

According to some embodiments of any of the embodiments described herein, the conditions comprise incubation at 37° C.

According to some embodiments of any of the embodiments described herein, the cells comprise at least two different cell types.

According to some embodiments of any of the embodiments described herein, the contacting is effected following culturing the cells of the cellularized engineered construct for a length of time such that the at least a portion of the cells interact biologically with one another.

According to some embodiments of any of the embodiments described herein, the cells comprise cells of connective tissue, muscle tissue, nervous tissue and/or epithelial tissue.

According to some embodiments of any of the embodiments described herein, the cells comprise endothelial cells and cardiomyocytes.

According to some embodiments of any of the embodiments described herein, the engineered cellularized construct is generated by 3D bioprinting.

According to some embodiments of any of the embodiments described herein, the engineered cellularized construct is generated by sequentially forming a plurality of layers on a receiving medium in a configured pattern corresponding to the shape of the engineered construct by 3D bioprinting, wherein for at least a few of the layers the forming is effected by dispensing of at least one bioink composition that comprises the ECM-based hydrogel and the cells.

According to some embodiments of any of the embodiments described herein, the dispensing is in accordance with a 3D printing data corresponding to the shape of the engineered construct.

According to some embodiments of any of the embodiments described herein, the dispensing is of at least two bioink compositions, at least one of the bioink compositions comprises the ECM-based hydrogel and a first type of cells, and at least another one of the bioink compositions comprises a second type of cells which is different from the first type of cells.

According to some embodiments of any of the embodiments described herein, the at least one bioink compositions further comprises an internal support material.

According to some embodiments of any of the embodiments described herein, the dispensing is further of a composition that provides an internal support material.

According to some embodiments of any of the embodiments described herein, the construct is a vascularized construct and the cells comprise endothelial cells.

According to some embodiments of any of the embodiments described herein, the construct is a vascularized construct and the second type of cells comprise endothelial cells.

According to some embodiments of any of the embodiments described herein, the at least bioink composition that comprises the second type of cells further comprises an internal support material.

According to some embodiments of any of the embodiments described herein, the dispensing is further of a composition that provides an external supporting medium.

According to some embodiments of any of the embodiments described herein, the method further comprises perfusing the cellularized engineered construct.

According to some embodiments of any of the embodiments described herein, the perfusing is effected subsequent to contacting the cellularized engineered construct with the reinforcing agent.

According to some embodiments of any of the embodiments described herein, the ECM-based hydrogel is derived from omental tissue.

According to some embodiments of any of the embodiments described herein, the cells are primary cells.

According to some embodiments of any of the embodiments described herein, the cells are differentiated ex vivo from pluripotent stem cells.

According to some embodiments of any of the embodiments described herein, the cells are induced pluripotent stem cells.

According to some embodiments of any of the embodiments described herein, the cells are mature cells.

According to an aspect of some embodiments of the present invention there is provided a cellularized engineered construct obtainable by the method as described herein in any of the respective embodiments and any combination thereof.

According to an aspect of some embodiments of the present invention there is provided a cellularized engineered construct comprising cells distributed within a chemically cross-linked ECM-based hydrogel, wherein the ECM-based hydrogel is chemically cross-linked by a biocompatible small-molecule reinforcing agent, as described herein in any of the respective embodiments and any combination thereof, that is capable of chemically interacting with the ECM-based hydrogel under conditions that maintain viability of the cells, and wherein a compressive modulus of the ECM-based hydrogel is higher by at least 10% than a compressive modulus of the ECM-based hydrogel which is not chemically cross-linked, wherein the construct is devoid of RPE cells.

According to some embodiments of any of the embodiments described herein, the biocompatible small-molecule reinforcing agent is chemically interacted with at least 10% of chemically compatible groups present in the ECM-based hydrogel before chemically interacting with the reinforcing agent, the chemically compatible groups are those that are capable of chemically interacting with the reinforcing agent under conditions that maintain viability of the cells.

According to some embodiments of any of the embodiments described herein, the chemically interacting effects cross-linking of the ECM-based hydrogel.

According to some embodiments of any of the embodiments described herein, the reinforcing agent is an oxidized, poly-aldehyde saccharide.

According to some embodiments of any of the embodiments described herein, the cells comprises at least two different cell types.

According to some embodiments of any of the embodiments described herein, the cells comprise cells of connective tissue, muscle tissue, nervous tissue or epithelial tissue.

According to some embodiments of any of the embodiments described herein, the cells comprise endothelial cells and cardiomyocytes.

According to some embodiments of any of the embodiments described herein, the construct is a vascularized construct and the cells comprise endothelial cells.

According to some embodiments of any of the embodiments described herein, the ECM-based hydrogel is derived from omental tissue.

According to some embodiments of any of the embodiments described herein, the cells are primary cells.

According to some embodiments of any of the embodiments described herein, the cells are differentiated ex vivo from pluripotent stem cells.

According to some embodiments of any of the embodiments described herein, the cells are iPSCs.

According to some embodiments of any of the embodiments described herein, the cells are mature cells.

According to an aspect of some embodiments of the present invention there is provided a cellularized engineered construct as described herein in any of the respective embodiments and any combination thereof is for use in treating a condition associated with a damaged tissue.

According to an aspect of some embodiments of the present invention there is provided a method of treating a condition associated with a damaged tissue in a subject in need thereof, the method comprising implanting the cellularized engineered construct as described herein in any of the respective embodiments and any combination thereof in the subject, thereby treating the condition associated with the damaged tissue.

According to some embodiments of any of the embodiments described herein, the method further comprises imaging the damaged tissue of the subject prior to the implanting so as to obtain 3D printing data for generating of the cellularized engineered construct.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A presents a schematic illustration of a method of reinforcing a 3D printed cardiac tissue according to exemplary embodiments of the present invention. Cardiac and endothelial cells are combined with hydrogels to create bio-inks, which are 3D printed to form a natively vascularized tissue. After allowing the cells to self-organize in a soft gel, a tissue-penetrating small molecule (a reinforcing agent) as described herein in any of the respective embodiments is introduced to homogenously reinforce the tissue.

FIG. 1B presents the chemical structures of an exemplary reinforcing agent according to some of the present embodiments.

FIGS. 2A-K presents the reinforcing effect of an exemplary reinforcing agent of on an exemplary ECM-based hydrogel. FIG. 2A presents a schematic illustration of a reinforcing process according to some embodiments of the present invention. Tissues are fabricated using 3D printing technology, which deposits cells and ECM fibers with random orientation (I); Cells are then allowed to self-organize and mature in a soft gel (II); and the engineered tissue is thereafter thoroughly reinforced by the diffusion of SOx into the tissue (III). FIG. 2B presents a schematic illustration of the reaction of the poly-aldehyde SOx with amine moieties present in collagen via “Click” chemistry to form imine bridges. FIG. 2C is a bar graph showing the viability of Primary human umbilical vein endothelial cells (HUVECs) grown in 2D culture containing various concentration of SOx for 48 hours. FIG. 2D presents comparative plots showing the shear thinning behavior of a non-reinforced hydrogel, and hydrogel reinforced in the present of 0.03 or 0.07% Sox, at 37° C. FIG. 2E is a bar graph showing the hydrogel bulk modulus with and without exposure to SOx. FIG. 2F is a bar graph showing the absorbance of a solution of ninhydrin that was allowed to react with the hydrogel, normalized to the control's value, indicating the portion of the amine groups in the hydrogel that reacted with SOx. FIGS. 2G and 2H present images showing the fiber morphology of ECM-based hydrogel before (FIG. 2G) and after (FIG. 2H) exposure to SOx. FIG. 2I is a bar graph showing the average pore size as calculated for the images shown in FIGS. 2G and 2H. FIG. 2J presents comparative plots showing the % of gel that remained after incubation in the presence of the SDS disintegrating detergent overnight. FIG. 2K presents comparative plots showing the % of the gel that remained after incubation in the presence of collagenase for 2 weeks.

FIGS. 3A-E presents the preparation and characterization of the cells for 3D bioprinting. FIGS. 3A-B present immunostaining images (FIG. 3A) and flow cytometry (FIG. 3B) of iPSCs characterization. FIGS. 3C-D present immunostaining images (FIG. 3C) and flow cytometry data (FIG. 3D), showing the induced pluripotent stem cell derived differentiated cardiomyocytes. FIG. 3E presents immunostaining data of primary endothelial cells, which formed a typical “cobblestone” pattern in 2D and formed tight junctions as judged by CD31 expression.

FIGS. 4A-F present the printing and characterization of a reinforced cardiac tissue. FIG. 4A is a schematic illustration of the finite elements modeling used to rationally design the geometry of blood vessels in the cardiac tissue. FIG. 4B are photographs showing the printed tissue. Support material provided external stabilization (yellow), and a cardiomyocyte-laden ink (represented in red) and endothelial cell-laden ink (represented in blue) were deposited layer by layer. FIG. 4C presents the localization of cells in the printed tissue. Cells were marked with a fluorescent cytopainter before printing. FIG. 4D presents images showing perfusion within the printed and subsequently reinforced tissue. FIG. 4E is an immunostaining photograph showing the cardiac cell morphology within the printed tissue. FIG. 4F presents calcium transients within the printed tissue.

FIGS. 5A-H presents data obtained in studying the reinforcement effect on the printed cardiac tissue. FIG. 5A are photographs showing subjecting the tissue to injection forces. FIG. 5B presents a SEM image of the hydrogel before injection. FIG. 5C presents a SEM image of the hydrogel after injection. FIG. 5D presents images showing post-injection perfusion. FIG. 5E presents confocal microscopy images of a top-view (top panel) and side-view (bottom panel) of the printed endothelial cells. FIG. 5F presents immunostaining image showing the cardiac cell morphology after injection.

FIG. 5G presents the recovery of the injected tissue. FIG. 5H presents calcium transients within the printed tissue post-injection.

### DESCRIPTION OF SPECIFIC EMBODIMENTS OF THE INVENTION

The present invention, in some embodiments thereof, relates to 3D bioprinting and, more particularly, but not exclusively, to formulations usable in 3D bioprinting of cellularized objects, to 3D bioprinting methods employing same, and to cellularized objects obtained thereby and uses thereof.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Despite advances in biomaterials engineering, a large gap remains between the weak mechanical properties that can be achieved with natural materials and the strength of synthetic materials. While many methods are known for chemically crosslinking collagen-based biomaterials, these techniques are toxic and are thus suitable only for crosslinking acellular structures, not pre-formed, fully cellularized tissues. Moreover, in many cases, applying a crosslinker (a cross-linking agent) into a polymeric biomaterial may result in a non-homogenous strengthening of the scaffold.

In a search for a novel methodology that overcomes these challenges above, the present inventors have designed a method for reinforcing an engineered cellularized tissue fabricated from ECM-based hydrogel to thereby provide an entire construct that is internally and externally reinforced in a homogenous, safe, and biocompatible manner.

The present inventors have established that incorporating a small reinforcing biomolecule in growth media after tissue assembly and during its maturation process allows it to penetrate deep into the entire engineered structure and significantly and safely increases the tissue's strength from within.

The present inventors have demonstrated the ability to manipulate the microenvironment of cultivated cells to meet the changing needs of engineered tissue. The reinforcement occurs as a post-fabrication step, which allows for the use of, inter alia, 3D printing technology to generate thick, fully cellularized, and, if needed, vascularized tissues. After tissue assembly and during the maturation process in a soft hydrogel, a small, tissue-penetrating reinforcer is deployed, leading to a significant increase in the tissue's mechanical properties. The tissue's robustness was demonstrated by injecting the tissue in a simulated minimally invasive procedure thereby showing that the tissue is functional and undamaged at the nano-, micro-, and macro-scales.

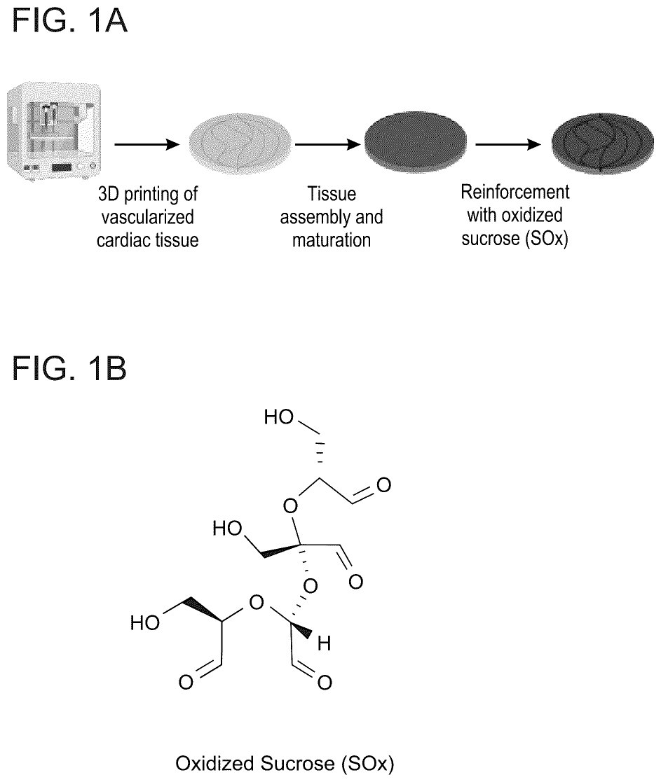

FIG. 1A presents a schematic illustration of a method of post-assembly reinforcement of a cellularized engineered tissue, according to some embodiments of the present invention.

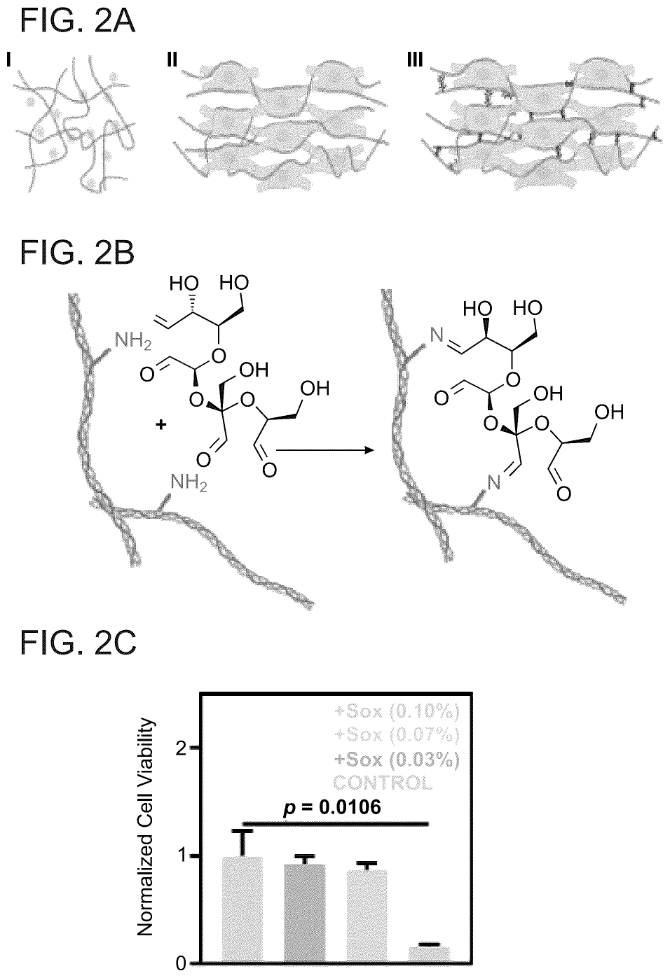

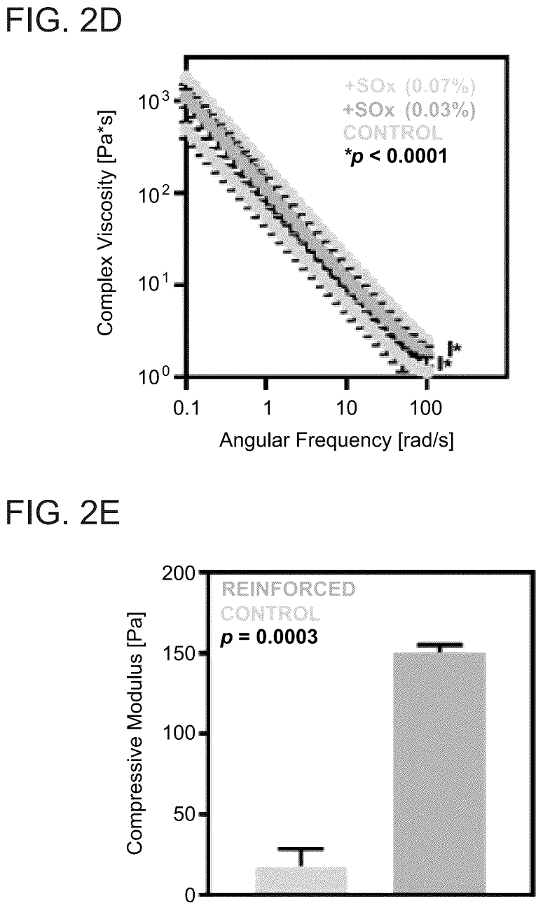

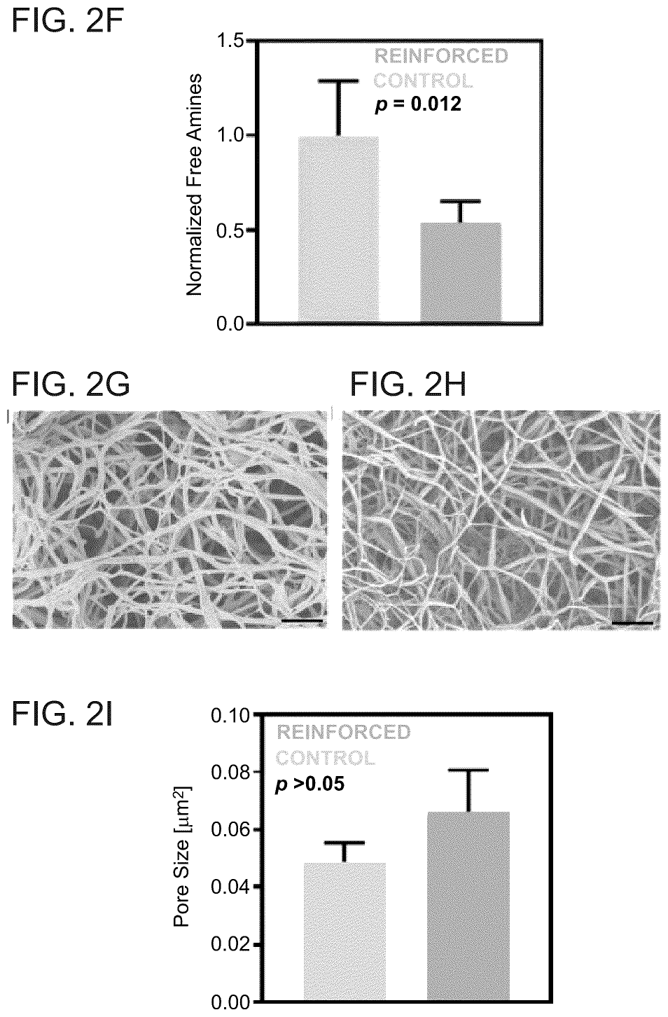

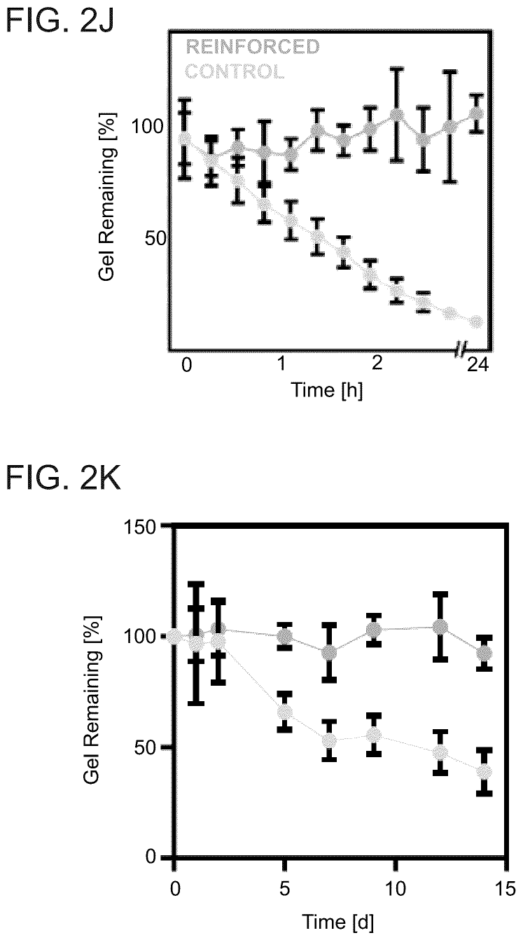

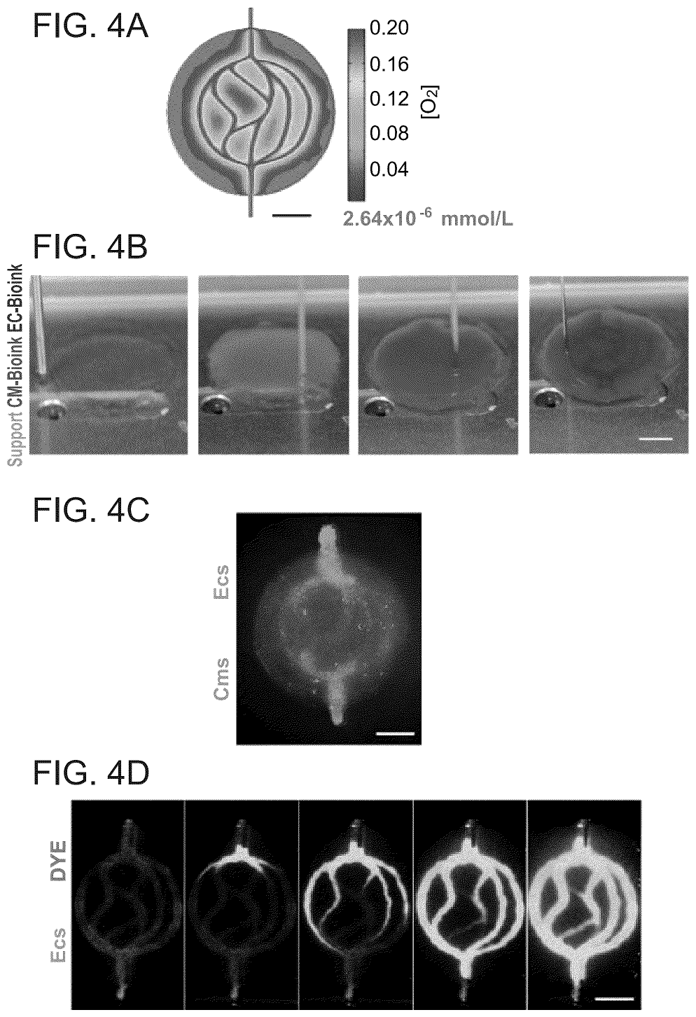

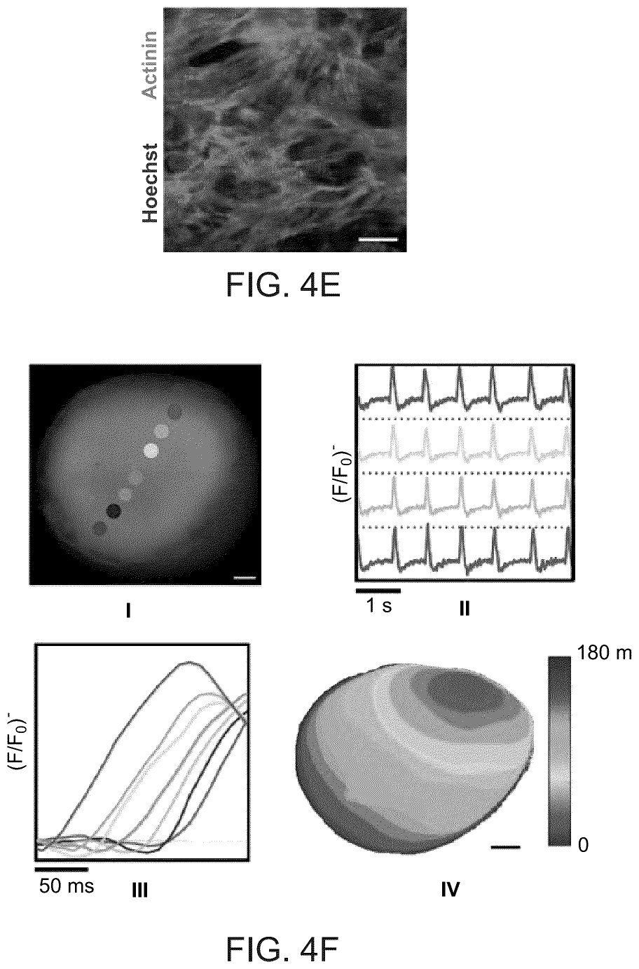

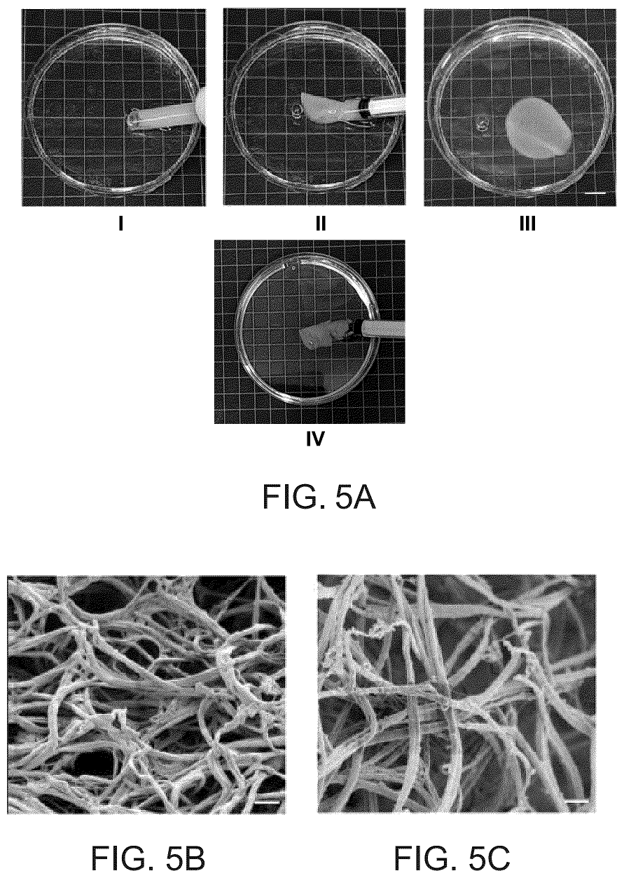

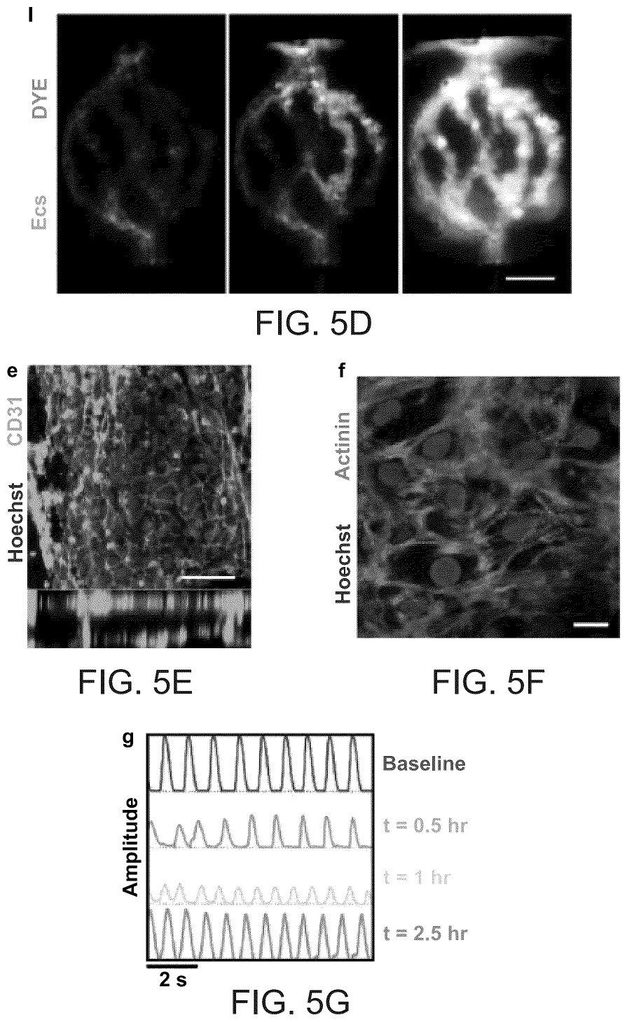

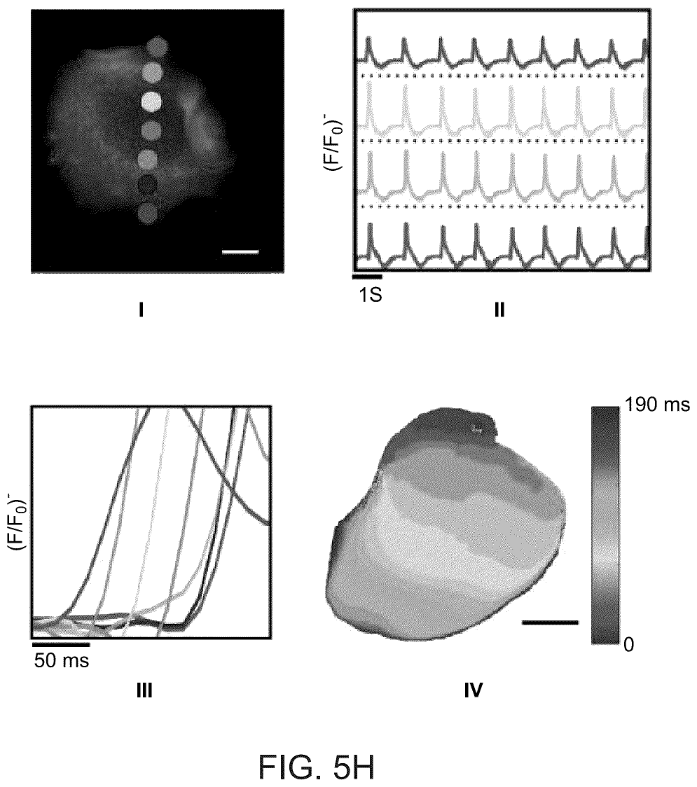

The present inventors have utilized the newly designed methodology for fabricating a cardiac tissue using 3D-printing technology and cellularized bioinks, ensuring a uniform distribution of cells and providing the tissues with a vascular tree ab initio. Immediately after printing, cells were allowed to self-assemble within the soft hydrogel (see, FIGS. 1A and 2A). Following the initial self-assembly of the tissue, and during the maturation phase, the entire construct was internally and externally reinforced in a homogenous, safe, and biocompatible manner (see, FIGS. 4A-F). These reinforced tissues could be subjected to significant stress without any deformation or adverse effect on their morphology and function (see, FIGS. 5A-H).

Embodiments of the present invention therefore relate to a newly designed methodology for providing reinforced engineered cellularized tissues, which employ a reinforcing agent as described herein in any of the respective embodiments, to reinforced engineered cellularized tissues obtained thereby and to uses thereof.

According to an aspect of the present invention, there is provided a method for reinforcing an engineered cellularized construct fabricated from extracellular matrix (ECM) hydrogel and cells. According to some of any of the embodiments described herein, the method utilized a biocompatible small-molecule reinforcing agent, such as described herein. According to some of any of the embodiments described herein, the biocompatible small-molecule reinforcing agent is capable of chemically interacting with the ECM-based hydrogel under conditions that maintain viability of the cells. According to some of any of the embodiments described herein, the method comprises contacting the engineered cellularized construct with the biocompatible small molecule reinforcing agent as described herein. According to some of any of the embodiments described herein, the biocompatible small-molecule reinforcing agent is capable of chemically interacting with the ECM-based hydrogel under conditions that maintain viability of the cells to thereby increase a compressive modulus of the ECM-based hydrogel by at least 10%, or at least 20%, or at least 50%, as described herein.

According to some of any of the embodiments described herein, the construct is devoid of retinal pigment epithelial (RPE) cells.

According to some of any of the embodiments described herein, the construct is devoid of photoreceptor cells.

According to some of any of the embodiments described herein, the construct is devoid of retinal pigment epithelial (RPE) cells and of photoreceptor cells.

According to some of any of the embodiments described herein, the construct does not form a retinal tissue.

According to another aspect of the present invention, there is provided a method of preparing a cellularized engineered construct, while employing a small molecule reinforcing agent as described herein. According to some of any of the embodiments described herein, the method is generally effected by generating the engineered cellularized construct, using a bioink composition that comprises an ECM-based hydrogel and cells, allowing the cells to interact and arrange within the generated engineered cellularized construct and subsequently contacting the cellularized engineered construct with a reinforcing agent as described herein. According to some of any of the embodiments described herein, generating the engineered cellularized construct is effected by culturing cells in the presence of an ECM-based hydrogel, so thereby provide the bioink composition; depositing the bioink composition, preferably in a configured pattern corresponding to the shape of the engineered construct; and culturing the cells of the engineered construct.

According to some of any of the embodiments described herein, subsequent to culturing the cells in the engineered cellularized construct, the method proceeds to contacting the cellularized engineered construct with a reinforcing agent as described herein in of the respective embodiments and any combination thereof. It will be appreciated that culturing may be continued following addition of the reinforcing agent, for example for at least 1, 2, 3, 4, 5, 6, 7 or more days. According to some of these embodiments, the contacting with the reinforcing agent is subsequent to part of the whole culturing process, and can be regarded as performed subsequent to initial culturing and during the culturing as a whole.

According to some of any of the embodiments described herein, the method is such that a compressive modulus of the ECM-based hydrogel upon contacting the cellularized engineered construct is higher by at least 10%, or at least 20%, or by at least 30%, or by at least 50%, or by at least 60%, or by at least 70%, or by at least 80%, or by at least 90%, or by at least 100%, compared to the compressive modulus of the hydrogel-based hydrogel before contacting the reinforcing agent, and/or compared to the same hydrogel-based hydrogel when the cellularized engineered construct is generated without contacting the reinforcing agent.

According to some embodiments, a compressive modulus of the ECM-based hydrogel is higher by at least 10%, or at least 20%, or by at least 30%, or by at least 50%, or by at least 60%, or by at least 70%, or by at least 80%, or by at least 90%, or by at least 100% than a compressive modulus of the same ECM-based hydrogel which is not chemically cross-linked, and which can be physically cross-linked as a result of the physically cross-linked fibrous network that provides the hydrogel.

Herein and in the art, the phrase “compressive modulus”, which is also referred to in the art compressive elastic modulus or compressive modulus of elasticity, describes a mechanical property that reflects the ability of a material to resist deformation under compressive loading, and accordingly is a measure of the material's stiffness in compression. When a material is subjected to a compressive force, it undergoes deformation or compression. The compressive modulus quantifies how much the material will deform under this compressive stress, is expressed in units of pressure. According to some embodiments, the compressive modulus is determined using a method as described in the Examples section that follows, by compressing the samples at a fixed rate and employing rheological measurements (using a rheometer).

The term “cellularized construct” (also referred to as a tissue) refers to a three-dimensional cellular aggregate in which at least a portion of the cells interact with one another and perform at least one tissue function.

Examples of tissues include, but are not limited to, connective tissue (e.g., areolar connective tissue, dense connective tissue, elastic tissue, reticular connective tissue, and adipose tissue), muscle tissue (e.g., skeletal muscle, smooth muscle and cardiac muscle), genitourinary tissue, gastrointestinal tissue, pulmonary tissue, bone tissue, nervous tissue, and epithelial tissue (e.g., simple epithelium and stratified epithelium), endoderm-derived tissue, mesoderm-derived tissue, and ectoderm-derived tissue.

The cellularized construct may be an organ, or a part thereof.

As used herein, “organ” means a collection of tissues joined into structural unit to serve a common function. Examples of organs include, but are not limited to, skin, urethra, conduit, ureter, bladder, fallopian tube, uterus, trachea, bronchus, lymphatic vessel, esophagus, stomach, gallbladder, small intestine, large intestine and colon.

In some embodiments, any vertebrate cell is suitable for inclusion in the engineered, cellularized constructs. In further embodiments, the cells are, by way of non-limiting examples, contractile or muscle cells (e.g., skeletal muscle cells, cardiomyocytes, smooth muscle cells, and myoblasts), connective tissue cells (e.g., bone cells, cartilage cells, fibroblasts, and cells differentiating into bone forming cells, chondrocytes, or lymph tissues), bone marrow cells, endothelial cells, skin cells, epithelial cells, breast cells, vascular cells, blood cells, lymph cells, neural cells, Schwann cells, gastrointestinal cells, liver cells, pancreatic cells, lung cells, tracheal cells, corneal cells, genitourinary cells, kidney cells, reproductive cells, adipose cells, parenchymal cells, pericytes, mesothelial cells, stromal cells, undifferentiated cells (e.g., embryonic cells, stem cells, and progenitor cells), endoderm-derived cells, mesoderm-derived cells, ectoderm-derived cells, and combinations thereof.

In one embodiment, the constructs are devoid of retinal pigment epithelial (RPE) cells. In another embodiment, the constructs are devoid of photoreceptors.

According to a particular embodiment, the cells are intact (i.e., whole), and preferably viable.

The cells may be primary cells, immortalized cells or derived from cell lines.

The cells may be fresh, frozen or preserved in any other way known in the art (e.g., cryopreserved).

In one embodiment, the cells used to fabricate the construct are genetically modified (e.g. to express a therapeutic agent or a detectable moiety) by any suitable method known in the art.

The cellularized construct may comprise one of more layers of cells. Each cell layer may be fabricated from a single cell type or a plurality of cell types. In one embodiment, the layer is a monolayer.

The cellularized construct may comprise at least two cell types of a single tissue (e.g. connective tissue, muscle tissue, nervous tissue or epithelial tissue). In one embodiment, the cellularized construct is a cardiac construct and the cells used to fabricate the construct comprise cardiomyoctyes and endothelial cells (and optionally smooth muscle cells).

For example, the cellularized construct may be a cardiac construct and comprise cardiomyocytes, endothelial cells and optionally fibroblasts.

As used herein, the term “cardiomyocytes” refers to fully or at least partially differentiated cardiomyocytes. Thus, cardiomyocytes may be derived from cardiac tissue or from stem cells (such as embryonic stem cells, induced pluripotent stem cells or adult stem cells, such as mesenchymal stem cells). Methods of differentiating stem cells along a cardiac lineage are well known in the art—[Muller-Ehmsen J, et al., Circulation. 2002; 105:1720-6; Zhang M, et al., J Mol Cell Cardiol. 2001; 33:907-21, Xu et al, Circ Res. 2002; 91:501-508, and U.S. Pat. Appl. No. 20050037489, the contents of which are incorporated by reference herein]. According to one embodiment the stem cells are derived from human stem cell lines, such as H9.2 (Amit, M. et al., 2000. Dev Biol. 227:271).

According to one embodiment the cardiomyocytes of the constructs are at least capable of spontaneous contraction. According to another embodiment, the cardiomyocytes of constructs of the present invention express at least one marker (more preferably at least two markers and even more preferably at least three markers) of early-immature cardiomyocytes (e.g. atrial natriuretic factor (ANF), Nkx2.5, MEF2C and α-skeletal actin). According to another embodiment, the cardiomyocytes of the constructs of the present invention express at least one marker (more preferably at least two markers and even more preferably at least three markers) of fully differentiated cardiomyocytes (e.g. MLC-2V, α-MHC, α-cardiac actin and Troponin I).

According to a particular embodiment, the construct is vascularized i.e. comprises at least one tubular structure generated from endothelial cells, and optionally fibroblasts.

The endothelial cells may be human embryonic stem cell (hESC)-derived endothelial cells (Levenberg, et al., Proc Natl Acad Sci USA (2002) 99, 4391-4396, the contents of which are incorporated by reference herein), iPSC-derived endothelial cells or primary endothelial cells cultured from e.g. human umbilical vein (HUVEC), or biopsy-derived endothelial cells such as from the aorta or umbilical artery. The endothelial cells of the constructs of the present invention may also be derived from humans (either autologous or non-autologous) e.g. from the blood or bone marrow. In addition the endothelial cells may be derived from other mammals, for example, humans, mice or cows. For example, endothelial cells may be retrieved from bovine aortic tissue. Preferably, the endothelial cells are not derived from the cardiac tissue from which the cardiac cells were isolated.

In one embodiment, human embryonic endothelial cells are produced by culturing human embryonic stem cells in the absence of LIF and bFGF to stimulate formation of embryonic bodies, and isolating PECAM1 positive cells from the population. HUVEC may be isolated from tissue according to methods known to those skilled in the art or purchased from cell culture laboratories such as Cambrex Biosciences or Cell Essentials.

Promotion of 3D endothelial structures may also be enhanced by addition of fibroblast cells (e.g. human embryonic fibroblasts) or other relevant mural cells (e.g., podocytes, astroblasts, pericytes, etc.). Fibroblasts may be isolated from tissue according to methods known to those skilled in the art (e.g. obtained from E-13 ICR embryos) or purchased from cell culture laboratories such as Cambrex Biosciences or Cell Essentials.

The cells of the cellularized construct may be derived from any organism including for example mammalian cells, (e.g., human, porcine), plant cells, algae cells, fungal cells (e.g., yeast cells), prokaryotic cells (e.g., bacterial cells).

According to a particular embodiment the cells are derived from (or comprise) stem cells—e.g., adult stem cells such as mesenchymal stem cells or pluripotent stem cells such as embryonic stem cells or induced pluripotent stem cells (iPSCs). The stem cells may be modified so as to undergo ex vivo differentiation prior to engineering of the construct or may be used as pluripotent stem cells and further differentiated in situ prior to implantation.

The phrase “embryonic stem cells” refers to embryonic cells which are capable of differentiating into cells of all three embryonic germ layers (i.e., endoderm, ectoderm and mesoderm), or remaining in an undifferentiated state. The phrase “embryonic stem cells” may comprise cells which are obtained from the embryonic tissue formed after gestation (e.g., blastocyst) before implantation of the embryo (i.e., a pre-implantation blastocyst), extended blastocyst cells (EBCs) which are obtained from a post-implantation/pre-gastrulation stage blastocyst (see WO 2006/040763), embryonic germ (EG) cells which are obtained from the genital tissue of a fetus any time during gestation, preferably before 10 weeks of gestation, and cells originating from an unfertilized ova which are stimulated by parthenogenesis (parthenotes).

It will be appreciated that commercially available stem cells can also be used according to some embodiments of the invention. Human ES cells can be purchased from the NIH human embryonic stem cells registry [Hypertext Transfer Protocol://grants (dot) nih (dot) gov/stem_cells/registry/current (dot) htm]. Non-limiting examples of commercially available embryonic stem cell lines are BG01, BG02, BG03, BG04, CY12, CY30, CY92, CY10, TE03, TE32, CHB-4, CHB-5, CHB-6, CHB-8, CHB-9, CHB-10, CHB-11, CHB-12, HUES 1, HUES 2, HUES 3, HUES 4, HUES 5, HUES 6, HUES 7, HUES 8, HUES 9, HUES 10, HUES 11, HUES 12, HUES 13, HUES 14, HUES 15, HUES 16, HUES 17, HUES 18, HUES 19, HUES 20, HUES 21, HUES 22, HUES 23, HUES 24, HUES 25, HUES 26, HUES 27, HUES 28, CyT49, RUES3, WA01, UCSF4, NYUES1, NYUES2, NYUES3, NYUES4, NYUES5, NYUES6, NYUES7, UCLA 1, UCLA 2, UCLA 3, WA077 (H7), WA09 (H9), WA13 (H13), WA14 (H14), HUES 62, HUES 63, HUES 64, CT1, CT2, CT3, CT4, MA135, Eneavour-2, WIBR1, WIBR2, WIBR3, WIBR4, WIBR5, WIBR6, HUES 45, Shef 3, Shef 6, BJNhem19, BJNhem20, SA001, SA001.

Induced pluripotent stem cells (iPSCs; embryonic-like stem cells), are cells obtained by de-differentiation of adult somatic cells which are endowed with pluripotency (i.e., being capable of differentiating into the three embryonic germ cell layers, i.e., endoderm, ectoderm and mesoderm). According to some embodiments of the invention, such cells are obtained from a differentiated tissue (e.g., a somatic tissue such as omentum) and undergo de-differentiation by genetic manipulation which re-program the cell to acquire embryonic stem cells characteristics. According to some embodiments of the invention, the induced pluripotent stem cells are formed by inducing the expression of Oct-4, Sox2, Kf14 and c-Myc/1-Myc in omental cells.

According to some of any of the embodiments described herein, the matrix of which at least a portion of the cellularized engineered construct is formed is an ECM-based hydrogel, as described herein in any of the respective embodiments and any combination thereof.

Herein and in the art, the term “hydrogel” describes a three-dimensional fibrous network containing at least 20%, typically at least 50%, or at least 80%, and up to about 99.99% (by mass) water or an aqueous solution. A hydrogel can be regarded as a material which is mostly water, yet behaves like a solid or semi-solid due to a three-dimensional interconnected solid-like fibrous network, within the liquid dispersing medium.

As used herein the phrase “fibrous network” refers to a set of connections formed between a plurality of fibrous components. Herein, the fibrous components are optionally composed of a plurality of polymeric chains, typically fibrillar polymeric chains, which can be made of polymeric biological materials (e.g., macromolecules) such as peptides, proteins, oligonucleotides and nucleic acids and/or of synthetic materials, preferably biocompatible polymers.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymeric chains, the type of the interaction between the polymeric chains (type of cross-linking), the degree of cross-linking (number of interconnected links between the chains), the aqueous media content and composition, and temperature.

The interconnecting links between the chains in the fibrous network can be chemical or physical, and are also referred to as chemical cross-linking and physical cross-linking, respectively.

By chemical cross-linking, it is meant the at least a portion of the chains composing the network are covalently linked to one another.

By physical cross-linking, it is meant that the chains composing the network are linked to one another either physically, for example, by means of entanglement, and/or chemically, via non-covalent interactions (e.g., electrostatic and/or hydrogen and/or aromatic interactions).

The degree of cross-linking describes the mol % of chains that are interconnected to one another chemically, via covalent bonds.

According to some embodiments, a hydrogel as described herein, prior to contacting the reinforcing agent, features a degree of crosslinking, as defined herein, of no more than 10%, or of no more than 5%, or of no more than 2%, or of no more than 1%, or null.

The tern “ECM-based hydrogel” refers to a hydrogel as described herein which comprises components of the extracellular matrix (ECM) including, but not limited to collagen, hyaluronic acid and elastin, that is, the fibrous network is composed mainly of fibrillar chains of ECM proteins. Typically, the hydrogel is derived from a tissue comprising ECM. According to embodiments of the invention, the ECM-based hydrogel is viscoelastic, thermoresponsive, has low swelling ratio and is biocompatible and degradable.

In one embodiment, the ECM-based hydrogel is not Matrigel.

In another embodiment, the ECM-based hydrogel is derived from decellularized mammalian tissue. Exemplary components of an ECM-based hydrogel derived from decellularized mammalian tissue include: collagen type I, II, III, IV, V, VI, laminin, elastin, fibronectin and glycosaminoglycans (sulfated and nonsulfated).

In additional embodiments, the ECM-based hydrogel is derived from decellularized mammalian tissue and is not enzymatically solubilized and neutralized to physiologic pH and temperature. Examples of such ECM-based hydrogels include decellularized human lipoaspirate, intervertebral disc and devitalized cartilage.

In additional embodiments, the ECM-based hydrogel is derived from decellularized mammalian tissue and is enzymatically solubilized and neutralized to physiologic pH and temperature.

Exemplary tissues which may be decellularized to form ECM-based hydrogels include, but are not limited to small intestinal submucosa (SIS), urinary bladder matrix (UBM), adipose tissue, bone, cartilage, heart, kidney, liver, lung, skeletal muscle, tendon, umbilical cord. According to a particular embodiment, the tissue is omentum.

In one embodiment, the tissue is porcine or bovine tissue. In another embodiment, the tissue is human tissue (e.g. human omental tissue).

Methods of decellularizing tissues are known in the art and are further described in Saldin et al., Acta Bimater, 2017, February 49, pages 1-15, Machluf et al (WO 2014/037942), Yang (EP 222191) and Badylak et al (US 2008/0260831) and Dvir et al., US 2020/0101198-A1) the contents of which are incorporated herein by reference.

Following decellularization, the decellularized tissue may be dehydrated e.g. lyophilized. The lyophilized, decellularized tissue may be cut into small pieces, e.g. crumbled, or milled into a powder and then subjected to proteolytic digestion. The digestion is effected under conditions that allow the proteolytic enzyme to digest and solubilize the ECM (e.g. by cleaving the telopeptide bonds of the collagen triple helix structure to unravel collagen fibril aggregates). Thus, according to one embodiment, the digestion is carried out in the presence of an acid (e.g. hydrochloric acid or acetic acid) so as to obtain a pH of about 1-4.

Proteolytic digestion according to this aspect of the present invention can be effected using a variety of proteolytic enzymes. Non-limiting examples of suitable proteolytic enzymes include trypsin, pepsin, collaganease and pancreatin which are available from various sources such as from Sigma (St Louis, MO, USA) and combinations thereof. Matrix degrading enzymes such as matrix metalloproteinases are also contemplated.

It should be noted that the concentration of the digestion solution and the incubation time therein depend on the type of tissue being treated and the size of tissue segments utilized and those of skilled in the art are capable of adjusting the conditions according to the desired size and type of tissue.

Preferably, the tissue segments are incubated for at least about 20 hours, more preferably, at least about 24 hours. Preferably, the digestion solution is replaced at least once such that the overall incubation time in the digestion solution is at least 40-48 hours.

Once the decellularized ECM is solubilized (when the liquid is homogeneous with no visible particles), the pH of the solution is increased so as to irreversibly inactivate the proteolytic enzyme (e.g. to about pH 7). The decellularized, solubilized tissue (e.g. omentum) may be stored at this stage at temperatures lower than 20° C.—for example 4° C. so that the decellularized ECM remains in solution.

Typically the ECM-based hydrogel has a DNA content per dry weight of hydrogel being less than 50 ng per dry weight of hydrogel, less than 40 ng per dry weight of hydrogel, or even less than 30 ng per dry weight of hydrogel.

According to still another embodiment, the diameter of the fibers in the ECM based hydrogel is between 5-500 nm (for example between 20-400 nm).

For a 1% gel, the storage modulus G′(t=0;Pa) may be between 10-20 for example 16-18. For a 1% gel, the storage modulus G′(t=0.5;Pa) may be between 100-200 for example 120-160. For a 1% gel, the storage modulus G′(t=0.95;Pa) may be between 100-200 for example 120-160. For a 1.5% gel, the storage modulus G′(t=0;Pa) may be between 10-50 for example 30-40. For a 1.5% gel, the storage modulus G′(t=0.5;Pa) may be between 200-500 for example 300-400. For a 1.5% gel, the storage modulus G′(t=0.95;Pa) may be between 200-500 for example 250-450.

For a 1% gel, the loss modulus G″ (t=0;Pa) may be between 5-20 for example 10-15. For a 1% gel, the loss modulus G″ (t=0.5;Pa) may be between 10-100 for example 20-50. For a 1% gel, the loss modulus G″ (t=0.95;Pa) may be between 10-100 for example 20-50. For a 1.5% gel, the loss modulus G″ (t=0;Pa) may be between 10-50 for example 20-40. For a 1.5% gel, the loss modulus G″ (t=0.5;Pa) may be between 10-100 for example 40-70. For a 1.5% gel, the loss modulus G″(t=0.95;Pa) may be between 10-100 for example 50-80.

The swelling ratio of the hydrogels of this aspect of the present invention are typically between 30-50, with the exact values depending on the length of time the hydrogel has been swollen and the percent precursor present in the hydrogel. Typically, the higher the precursor concentration in the hydrogel, the lower the swelling ratio.

In some embodiments, the ECM-based hydrogel is a thermoreversible gel (also known as thermo-responsive gel or thermogel). In some embodiments, a suitable thermoreversible hydrogel is aa liquid at room temperature. In some embodiments, a suitable thermoreversible hydrogel is a solid at physiological temperature. In specific embodiments, the gelation temperature (Tgel) of a suitable hydrogel is about 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., including increments therein. In certain embodiments, the Tgel of a suitable hydrogel is about 10° C. to about 40° C. In further embodiments, the Tgel of a suitable hydrogel is about 20° C. to about 30° C.

The engineered constructs may be fabricated using any method known in the art.

In one embodiment, the cells are combined with the ECM-based hydrogels and seeded on a solid or semi-solid scaffold. The architecture and 3D shape of the scaffold ultimately dictates the size and shape of the construct.

As used herein, the term “scaffold” refers to synthetic scaffolds such as polymer scaffolds, and non-synthetic scaffolds such as pre-formed extracellular matrix layers, dead cell layers, and decellularized tissues, and any other type of pre-formed scaffold that is integral to the physical structure of the engineered tissue and/or organ and not able to be removed from the tissue and/or organ without damage/destruction of said tissue and/or organ.

In some embodiments, at least one layer of the constructs is bioprinted. In other embodiments, the engineered constructs are entirely bioprinted, that is, are generated by bioprinting, in particular 3D bioprinting, as described herein in any of the respective embodiments and any combination thereof.

A bioprinting method and a corresponding system can be any of the methods and systems known in the art for performing additive manufacturing. A suitable method and system can be selected upon considering its printing capabilities, which include resolution, deposition speed, scalability, bio-ink compatibility and ease-of-use.

Exemplary suitable bioprinting systems usually contain a temperature-controlled material handling with a dispensing system and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote hardening or solidification of the formed layers and/or a humidifier, can also be included in the system. There are printers that use multiple dispensing heads to facilitate a serial dispensing of several materials.

3D bioprinting is an additive manufacturing methodology which uses biological materials, optionally in combination with chemicals and/or cells, that are printed layer-by-layer with a precise positioning and a tight control of functional components placement to create a 3D structure.

Inherent to 3D printing in general is that the mechanical properties of the printing media (the dispensed material; bioink) are different from the post-printed cured (hardened; solidified) material.

Different technologies have been developed for 3D bioprinting, including 3D Inkjet printing, Extrusion printing, Laser-assisted printing, digital light processing, and Projection stereolithography [see, for example, Murphy S V, Atala A, Nature Biotechnology. 2014 32(8).; Miller J S, Burdick J. ACS Biomater. Sci. Eng. 2016, 2, 1658-1661]. Each technology has its different requirements for the dispensed building material (also referred to herein as printing media), which is derived from the specific application mechanism and the curing/gelation process required to maintain the 3D structure of the scaffold post printing.

The following describes embodiments of additive manufacturing processes and methodologies for which the method as described herein can be employed.

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object. According to some embodiments of this aspect, formation of each layer is effected by dispensing at least one uncured building material, and exposing the dispensed building material to a curing condition to thereby form a hardened (cured; solidified) material. According to some embodiments of this aspect, formation of each layer is effected by exposing a layer of uncured building material to a curing condition, and the method is effected by sequentially exposing, in a layer-wise manner, an uncured building material to a curing condition.

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s) or bioink composition(s), and optionally also uncured materials used to form a support, namely uncured support material formulations. The building material can also include non-curable materials that preferably do not undergo (or are not intended to undergo) any change during the process, for example, biological materials or components and/or other agents or additives as described herein.

The building material that is used to sequentially form the layers as described herein is also referred to herein interchangeably as “printing medium” or “bioprinting medium” “bioink composition”, “bio-ink composition”, “bio-ink” or “bioink”.

An uncured building material can comprise one or more modeling material formulations, and can be utilized such that different parts of the object are made upon hardening of different modeling formulations, and hence are made of different hardened modeling materials or different mixtures of hardened modeling materials.

The method of the present embodiments manufactures three-dimensional objects in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different biological component; or each containing a different curable material; or each containing a different concentration of a curable material, and/or different support material formulations). Thus, different target locations can be occupied by different building materials (e.g., a modeling formulation and/or a support formulation, as defined herein).

The final three-dimensional object is made of the hardened modeling material or a combination of hardened modeling materials or a combination of hardened modeling material/s and support material/s or modification thereof. All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

An exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD). In some embodiments, the object is bio-printed in a size and shape that corresponds to a subject's anatomy. These parameters can be obtained, for example, from imaging data, such that the method can further comprise acquiring an imaging data for a size and shape of the object, and bioprinting the object in accordance with the imaging data. The imaging data can be of a subject to be treated with the object or of an exemplary data, so as to serve as a computed model for the additive manufacturing.

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer.

In some embodiments, the receiving medium comprises a sacrificial hydrogel or other biocompatible material as a mold to embed the printed object, and is thereafter removed by chemical, mechanical or physical (e.g., heating or cooling) means. Such sacrificial hydrogels can be made of, for example, a Pluronic material or of Gelatin. An exemplary sacrificial hydrogel is made of gelatin microparticles. An exemplary sacrificial hydrogel is made of alginate, xanthan gum, and gluconic acid δ-lactone (for example, a sacrificial external support referred to herein as “Granula”). A composition that provides a sacrificial hydrogel as a receiving medium is also referred to herein as a composition that provides an external supporting material (also referred to herein as an external supporting medium). In some embodiments, a sacrificial hydrogel as described herein is non-cellularized.

Once the uncured building material is dispensed on the receiving medium according to the 3D data, the method optionally and preferably continues by hardening the dispensed formulation(s). In some embodiments, the process continues by exposing the deposited layers to a curing condition. Preferably, the curing condition is applied to each individual layer following the deposition of the layer and prior to the deposition of the previous layer.

As used herein throughout, the term “curing” describes a process in which a formulation is hardened. The hardening of a formulation typically involves an increase in a viscosity of the formulation and/or an increase in a storage modulus of the formulation (G′). In some embodiments, a formulation which is dispensed as a liquid becomes solid or semi-solid (e.g., gel) when hardened. A formulation which is dispensed as a semi-solid (e.g., soft gel) becomes solid or a harder or stronger semi-solid (e.g., strong gel) when hardened.

The term “curing” as used herein encompasses, for example, polymerization of monomeric and/or oligomeric materials and/or (e.g., physical) cross-linking of polymeric chains (for example, ECM protein chains). The product of a curing reaction can therefore be a (e.g., physically) cross-linked material. This term, as used herein, encompasses also partial curing, for example, curing of at least 20% or at least 30% or at least 40% or at least 50% or at least 60% or at least 70% of the formulation, in addition to curing of 100% of the formulation.

Herein, the phrase “a condition that affects curing” or “a condition for inducing curing”, which is also referred to herein interchangeably as “curing condition” or “curing inducing condition” describes a condition which, when applied to a formulation that contains a curable material, induces a curing as defined herein. Such a condition can include, for example, application of a curing energy, as described hereinafter to the curable material(s), and/or simple solidification at ambient or physiological environment. In some embodiments, the condition includes a temperature change. Thus, in some embodiments, the bioink when used for printing is in a liquid form, e.g. printed at a temperature below 37° C., e.g., lower than 30, or lower than 25, or lower than 20, or at an ambient temperature (e.g., room temperature of about 25° C.), or at lower temperatures, typically upon cooling the system to a temperature lower than 20, or lower than 15, or lower than 10, or lower than 5, typically 4 or 0, ° C. The bioink hardens (or jellifies) at a temperature of 37° C., such that the curing condition comprises exposing the deposited hydrogel to a temperature of about 37° C., by, e.g., heating. Some embodiments contemplate the fabrication of an object by depositing or dispensing different formulations from different dispensing heads. These embodiments provide, inter alia, the ability to select formulations from a given number of formulations and define desired combinations of the selected formulations and their properties.

According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

In addition to the dispensing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for heating or cooling the dispensed building material or for contacting it with a reagent that promotes curing.

According to the present embodiments, the additive manufacturing method described herein is for bioprinting a biological object.

As used herein, “bioprinting” means practicing an additive manufacturing process while utilizing one or more bio-ink formulation(s) that comprise(s) biological components, as described herein, via a methodology that is compatible with an automated or semi-automated, computer-aided, additive manufacturing system as described herein (e.g., a bioprinter or a bioprinting system).

In the context of bioprinting, an uncured building material comprises at least one modeling formulation that comprises one or more biocompatible or biological components or materials (e.g., an ECM-based hydrogel as described herein and/or cells as described herein), and is also referred to herein and in the art as “bioink” or “bioink formulation” or “bioink composition”.

In some embodiments, the bioprinting comprises sequential formation of a plurality of layers of the uncured building material in a configured pattern, preferably according to a three-dimensional printing data, as described herein. At least one, and preferably most or all, of the formed layers (before hardening or curing) comprise(s) one or more biological component(s) as described herein (e.g., an ECM-based hydrogel and/or cells as described herein). Optionally, at least one of the formed layers (before hardening or curing) comprises one or more non-biological curable materials, and/or non-curable biological or non-biological components, preferably biocompatible materials which do not interfere (e.g., adversely affect) with the biological and/or structural features of the biological components in the printing medium and/or bio-ink.

In some embodiments, the components in the bioink or the printing medium, e.g., non-curable and curable materials, and/or the curing condition applied to effect curing, are selected such that they do not significantly affect structural and/or functional properties of the biological components in the bio-ink or printing medium.

In some of any of the embodiments described herein, the building material (e.g., the printing medium, a bioink or bio-ink) comprises modeling material formulation(s) (e.g., one or more bioink composition(s) as described herein) and optionally one or more support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bioink composition.

In some embodiments, a bioprinting system for effecting a bioprinting process/method as described herein is configured so as to allow formation of the layers under conditions that do not significantly affect structural and/or functional properties of the biological components in the bio-ink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

In some of any of the embodiments described herein, the additive manufacturing process (bioprinting) is performed while applying a shear force that does not adversely affect structural and/or functional properties of biological components (e.g., cells). Applying the shear force can be effected by passing the building material (e.g., at least a modeling material formulation that comprises a biological component as described herein; a bioink composition) through the dispensing head, and is to be regarded also as subjecting the building material to shear force.

Some embodiments of the present invention allow to perform AM bioprinting processes under conditions that do not affect the functional and/or structural features of biological components included in the bio-ink (e.g., at low shear force and room temperature or a physiological temperature), while maintaining the required fluidity (a viscosity that imparts fluidity, e.g., lower than 10,000 centipoises or lower than 5,000 centipoises, or lower than 2,000 centipoises), and while further maintaining the curability of the dispensed building material.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and ease-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials. Generally, bioprinting can be effected using any of the known techniques for additive manufacturing. The following lists some exemplary additive manufacturing techniques, although any other technique is contemplated.

3D Inkjet printing: 3D Inkjet printing is a common type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates. A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

Extrusion printing: This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic or mechanical dispensing systems

Stereolithography (SLA) and Digital Light Processing (DLP): SLA and DLP are additive manufacturing technologies in which an uncured building material in a bath or vat is converted into hardened material(s), layer by layer, by selective curing using a light source while the uncured material is later separated/washed from the hardened material. SLA is widely used to create models, prototypes, patterns, and production parts for a range of industries including for Bioprinting. DLP differs from laser-based SLA in that DLP uses a projection of ultraviolet (UV) light (or visible light) from a digital projector to flash a single image of the layer across the entire uncured material at once. One of the key components of DLP is a digital micronirror device (DMD) chip, which is typically composed of an array of reflective aluminum micromirrors that redirect incoming light from the UV source to project an image of a designed pattern. For achieving a high-resolution structure, parameters such as the curing time of each layer, layer thickness, and intensity of the UV light should be tuned, for example, by controlling the concentration and types of the curable materials, the photoabsorber and/or the photoinitiator.

Laser-assisted printing: Laser-assisted printing technique, in the version adopted for 3D bioprinting, is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate. Laser assisted printing is compatible with a series of viscosities and can print mammalian cells without affecting cell viability or cell function. Cells can be deposited at a density of up to 10⁸ cells/ml with microscale resolution of a single cell per drop.

Electrospinning: Electrospinning is a fiber production technique, which uses electric force to draw charged threads of polymer solutions, or polymer melts.

According to some of any of the embodiments described herein, the additive manufacturing (bioprinting) is an extrusion-based bioprinting, as described herein.

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

In some embodiments, the engineered constructs are comprised essentially of, or formed by using, the one or more bioink compositions (i.e. cellular material and ECM-based hydrogels) prior to reinforcing.

In various further embodiments, the cell-comprising portions of the engineered constructs consist of 30, 35, 40, 45, 50, 55, 60, 65, 70, 75, 80, 85, 90, 95, 96, 97, 98, 99, 99.5, 99.9, and 100% cellular material, including increments therein, prior to reinforcing. In one embodiment, at least 20%, 30%, 40%, 50%, 60%, 70%, 80%, 90% of the cells of the construct are mature cells prior to contacting with the reinforcing agent. In other various embodiments, the cell-comprising portions of the engineered constructs consist of 30, 35, 40, 45, 50, 55, 60, 65, 70, 75, 80, 85, 90, 95, 96, 97, 98, 99, 99.5, 99.9, and 100% cellular material, including increments therein, at the time of implantation. In some embodiments, the engineered constructs are cohered and/or adhered aggregates of cells prior to reinforcing (i.e. the constructs are cultured in a culture medium for a length of time such that at least a portion of the cells interact biologically with each other and are cohered and/or adhered prior to reinforcing).

In some embodiments, a portion of the non-cellular components (e.g., an internal and/or external support material as described herein) are removed prior to reinforcing. In further embodiments, the non-cellular components (e.g., an internal and/or external support material as described herein) are removed by physical, chemical, or enzymatic means. In some embodiments, a proportion of the non-cellular components (e.g., an internal and/or external support material as described herein) remains associated with the cellular components at the time of reinforcing. In some embodiments, the non-cellular components are selected from a group that includes: hydrogels, surfactant polyols, thermo-responsive polymers, hyaluronates, alginates, collagens, or other biocompatible natural or synthetic polymers.

In one embodiment, the bioink comprises a single-cell suspension in the ECM-based hydrogel. In another embodiment, the bioink comprises clusters of cells in the ECM based hydrogel.

In some embodiments, the bio-ink further comprises an agent that encourages cell adhesion.

In some embodiments, the bio-ink further comprises an agent that inhibits cell death (e.g., necrosis, apoptosis, or autophagocytosis). In some embodiments, the bio-ink further comprises an anti-apoptotic agent. Agents that inhibit cell death include, but are not limited to, small molecules, antibodies, peptides, peptibodies, or combination thereof. In some embodiments, the agent that inhibits cell death is selected from: anti-TNF agents, agents that inhibit the activity of an interleukin, agents that inhibit the activity of an interferon, agents that inhibit the activity of an GCSF (granulocyte colony-stimulating factor), agents that inhibit the activity of a macrophage inflammatory protein, agents that inhibit the activity of TGF-B (transforming growth factor B), agents that inhibit the activity of an MMP (matrix metalloproteinase), agents that inhibit the activity of a caspase, agents that inhibit the activity of the MAPK/JNK signaling cascade, agents that inhibit the activity of a Src kinase, agents that inhibit the activity of a JAK (Janus kinase), or a combination thereof. In some embodiments, the bio-ink comprises an anti-oxidant.

In some embodiments, the bio-ink further comprises an extrusion compound (i.e., a compound that modifies the extrusion properties of the bio-ink). Examples of extrusion compounds include, but are not limited to gels, hydrogels, peptide hydrogels, amino acid-based gels, surfactant polyols (e.g., Pluronic F-127 or PF-127), thermo-responsive polymers, hyaluronates, alginates, extracellular matrix components (and derivatives thereof), collagens, other biocompatible natural or synthetic polymers, nanofibers, and self-assembling nanofibers.

In some embodiments of any of the embodiments described herein, the bio-ink composition is devoid of a curing agent that promotes chemical cross-linking of the hydrogel components and/or of curable materials that can undergo chemical cross-linking upon exposure to a curing condition (e.g., a temperature change, for example, heating). By “devoid of” it is meant less than 5%, or less than 2%, preferably less than 1%, or less than 0.5%, or less than 0.1%, or less than 0.05%, or less than 0.01%, or null.

In some embodiments of any of the embodiments described herein, the bio-ink comprises one or more bioink compositions that comprise an ECM-based hydrogel and cells and/or as described herein in any of the respective embodiments, and the dispensing is of the one or more bioink compositions. In some embodiments, the bioink (printing medium; building material formulations) further comprises a composition that provides an internal support material. In some embodiments, the internal support material comprises a non-cellular sacrificial material or components (which can be removed after the bioprinting process as described herein). In exemplary embodiments, the internal support material comprises materials such as Pluronic acid and/or gelatin. In exemplary embodiments, it comprises gelatin microparticles as described herein.

According to embodiments of the present invention, a reinforcing agent is a biocompatible small molecule that is capable of penetrating a cellularized engineered tissue and thereby distribute substantially homogeneously throughout the tissue, both internally and externally. According to some embodiments, the biocompatible small-molecule reinforcing agent is capable of chemically interacting with one or more materials that form the construct matrix of the engineered tissue. According to some embodiments, the biocompatible small-molecule reinforcing agent is capable of chemically interacting with the materials that form the matrix under conditions that maintain viability of the cells, to thereby provide a chemically cross-linked matrix. According to some embodiments, the reinforcing agent acts as a cross-linking agent, which chemically cross-link polymeric chains in the matrix (e.g., protein chains in an ECM-based hydrogel as described herein).

By “biocompatible” it is meant that it poses limited risk of injury or toxicits to organisms that it contacts.

By “small molecule” it is meant that the molecule is spatially arranged such that it can penetrate through the pores of the matrix used to construct the tissue, that is, its volume is lower by at least 5%, or by at least 10%, and preferably much lower, than the average pore size of the matrix. According to some embodiments, the reinforcing has a molecular weight of no more than 1,000 grams/mol, or no more than 80 grams/mol, or no more than 600 grams/mol, or no more than 500 grams/mol, or no more than 400 grams/mol, or no more than 300 grams/mol, or no more than 200 grams/mol, and can range, for example, from 50 to 1,000, or from 100 to 1,000, or from 50 to 800, or from 100 to 800, or from 50 to 600 or from 100 to 600, or from 50 to 500, or from 100 to 500, or from 50 to 400, or from 100 to 400, or from 200 to 1,000 or from 200 to 800, or from 200 to 700, or from 200 to 600, or from 200 to 500, or from 200 to 400, or from 200 to 300, grams/mol, including any intermediate values and subranges therebetween.

According to some of any of the embodiments described herein, the reinforcing agent is capable of chemically interacting with one or more of the materials that compose the ECM-based hydrogel (e.g., one or more ECM proteins) used to construct the tissue under conditions that do not affect the viability of the cells within the matrix and the integrity of the matrix (e.g., mechanical and morphological properties of the cellularized matrix).

By “chemically interacting” it is meant that the reinforcing agent is capable of forming one or more chemical bonds with one or more functional groups of one or more of the materials that compose the matrix, whereby the chemical bonds can be hydrogen bonds, electrostatic bonds, Van-der-Waals bonds and/or covalent bonds. Preferably, the reinforcing agent is capable of forming covalent bonds with the one or more materials that form the matrix, that is, can be covalently attached to the matrix under the indicated conditions. The chemical interaction (covalent bond formation) is with free chemically compatible groups present in the matrix, that can interact with the reinforcing agent as described herein, under conditions that maintain viability of the cells, as described herein.

According to some of any of the embodiments described herein, the biocompatible small-molecule reinforcing agent is capable of chemically interacting, e.g., form covalent bonds, with at least 10%, at least 20%, at least 30%, at least 40% or at least 50%, of respective chemically compatible functional groups present in the ECM-based hydrogel before chemically interacting with said reinforcing agent.

Suitable conditions that do not affect the viability of the cells include a temperature around a physiological temperature, or otherwise a temperature that do not affect the viability of the cells; a lack of chemical reagents or solvents that may affect the viability of the cells; and a lack of physical means that may affect the viability of the cells and/or the integrity of a tissue formed of the cells and/or the integrity of the matrix. Preferably, the conditions include incubating the cellularized engineered tissue with an aqueous solution (e.g., a culturing medium) that comprises the reinforcing agent at a temperature around a physiological temperature (about 37° C.).

According to some embodiments of the present invention, the reinforcing agent is capable of interacting with a material that forms the matrix (e.g., an ECM protein) via a Click reaction.

Herein and in the art, a Click reaction describes a class of highly efficient, selective, and modular chemical reactions characterized by their orthogonality (compatibility with various functional groups) and high yields under mild reaction conditions.

An exemplary Click reaction according to the present embodiments is a Schiff-base reaction between an aldehyde and an amine-containing moiety such as amine, hydrazine, hydrazide and the like, as these terms are defined herein. This reaction can be carried out under mild conditions that do not affect the protein's essential characteristics (see, for example, Jansen E. F. and Olson A. C, Arch. Biochem. Biophys, 1969, 129(1), pp. 221-7 and U.S. Pat. No. 4,904,592).

According to some the present embodiments, the reinforcing agent features at least one, and preferably two or more, groups that are capable of participating in a Click reaction, e.g., Schiff-base reaction, with free chemically compatible groups in the ECM-based matrix.

According to some the present embodiments, the reinforcing agent features at least two, or preferably more, aldehyde group(s), and is therefore capable of forming covalent bonds with free chemically-compatible groups of the one or more materials used to form the matrix, for example, free amine groups, via a Schiff-base Click reaction. According to some embodiments of the present invention, the reinforcing agent is a polyaldehyde.

As used herein, the term “polyaldehyde” describes a compound that has at least two free aldehyde groups, as this term is defined herein.

Polyaldehydes can readily interact with various groups via “Schiff-base” chemistry, to form imine bonds, under mild conditions.

Free amine groups are typically included in protein-based matrices, such as ECM-based matrices as described herein, as functional groups derived from side chains of certain amino acid residues, functional groups derived from the N-terminus or the C-terminus of a protein, and/or functional groups derived from residues that result from natural post-translational modification processes.

Free amine groups may form a part of functional moieties such as lysine residues present on the surface of ECM proteins such as collagen.

A polyaldehyde that participate in a Schiff-base reaction can act as a cross-linking agent, by being covalently attached to one or more protein chains that form the matrix.

According to some of any of the embodiments described herein, the reinforcing agent is a biocompatible small molecule compounds, as defined herein, which features one or more, preferably two or more aldehyde groups.

According to some of these embodiments, the reinforcing agent (featuring two or more aldehyde groups, e.g., a polyaldehyde) is capable of interacting with free amine groups in the ECM-based hydrogel, via Schiff-base chemistry as described herein (a Click chemistry), to thereby form covalent bonds. According to some embodiments, the reinforcing agent is capable of chemically interacting with at least 10, at least 20, at least 30, at least 40 or at least 50, %, or more, of free amine groups present in the ECM-based hydrogel (before chemically interacting with the reinforcing agent). According to some of these embodiments, the chemical interactions with the free amine groups, results in cross-linking the hydrogel by means of covalent attachment to two or more protein chains in the ECM-based hydrogel.

According to some of any of the embodiments described herein, the reinforcing agent is a modified saccharide, that features one or more, preferably two or more, aldehyde groups, that is, it is a saccharide of which one or more hydroxy groups have been oxidized and thereby converted to aldehyde(s). Such a modified saccharide is also referred to herein and in the art as an oxidized saccharide.

The term “saccharide” as used herein encompasses monosaccharides, disaccharides and oligosaccharides. The term “monosaccharide”, as used herein and is well known in the art, describes a simple form of a sugar that consists of a single saccharide molecule, which can be open-chain or cyclic (e.g., pyranose- or furanose-based), and which cannot be further decomposed by hydrolysis. Most common examples of monosaccharides include glucose (dextrose), fructose, galactose, and ribose. Monosaccharides can be classified according to the number of carbon atoms of the carbohydrate, i.e., triose, having 3 carbon atoms such as glyceraldehyde and dihydroxyacetone; tetrose, having 4 carbon atoms such as erythrose, threose and erythrulose; pentose, having 5 carbon atoms such as arabinose, lyxose, ribose, xylose, ribulose and xylulose; hexose, having 6 carbon atoms such as allose, altrose, galactose, glucose, gulose, idose, mannose, talose, fructose, psicose, sorbose and tagatose; heptose, having 7 carbon atoms such as mannoheptulose, sedoheptulose; octose, having 8 carbon atoms such as 2-keto-3-deoxy-manno-octonate; nonose, having 9 carbon atoms such as sialose; and decose, having 10 carbon atoms. Monosaccharides are the building blocks of oligosaccharides and disaccharides like sucrose (common sugar).

The term “disaccharide” describes a compound two monosaccharide units, which can be the same or different, covalently bound to one another, typically via a glucosyl bond.

The term “oligosaccharide” as used herein describes a compound that comprises three or more monosaccharide units, as these are defined herein, which can be the same or different. Preferably, the oligosaccharide comprises 3-6 monosaccharides units.

According to some of any of the embodiments described herein, the reinforcing agent is an oxidized saccharide, as defined herein, which features at least two, at least three, at least four, or more aldehyde groups; and/or which has been oxidized so as to covert at least one, preferably at least two, at least three or at least four, of its hydroxy groups, into aldehyde groups.

Methods of generating oxidized saccharides are well-known in the art. An exemplary such method is described in the Examples section that follows.

According to an exemplary embodiment, the reinforcing agent is an oxidized disaccharide, such as an oxidized sucrose (SOx), the structure of which is presented in FIG. 1A.

It is to be noted that an oxidized saccharides can be selected so as to provide a desired reinforcement effect, in accordance with the materials used to form the matrix of the engineered tissue. For example, oxidized monosaccharides, or oxidized disaccharides featuring a lower molecular weight can be used to reinforce matrices that feature relatively small pores, and oxidized oligosaccharides, or oxidized monosaccharides and/or disaccharides featuring a higher molecular weight can be used to reinforce matrices that feature relatively voluminous pores.

Similarly, other compounds which feature a plurality of aldehyde groups (polyaldehydes), and which are biocompatible small molecules, can be used, and can be selected in accordance with the reinforced matrix, as above.

According to the present embodiments, the reinforcing agent is contacted with the cellularized engineered construct, subject to generation of the construct (e.g., following bioprinting of the construct, as described herein in any of the respective embodiments, or otherwise depositing a bio-ink composition as described herein, and allow it to harden, for example, by exposing to a suitable condition that promotes hardening or curing, as described herein).

According to some embodiments, the reinforcing agent is contacted with the construct under conditions as described herein.

According to some embodiments, the contacting is with a solution (e.g., aqueous solution) that comprises the reinforcing agent.

According to some embodiments, the contacting is effected by adding the reinforcing agent to a culturing medium, and subjecting the culturing medium and the construct to conditions as described herein, to thereby promote chemical interaction of the reinforcing agent with the construct. In some embodiments, the conditions comprise subjecting the culturing medium and the construct to incubation at 37° C.

According to some embodiments, the concentration of the reinforcing agent in the solution or the culturing medium is such that do not affect the viability of the cells in the construct. In some embodiments, the concentration of the reinforcing agent in the solution (e.g., culturing medium) ranges from about 0.01 to about 10%, or from about 0.01 to about 5%, or from about 0.01 to about 1%, or from about 0.01 to about 0.5%, or from about 0.01 to about 0.3%, or from about 0.01 to about 0.1%, by weight, of the total weight of the solution.

According to some embodiments of the present invention, the contacting is effected for a time period that ranges from about 1 hour to about several days (e.g., 2-14 days), or from about 12 hours to about several days, or from about 24 hours to about several days, including any intermediate values and subranges therebetween.

According to some embodiments, the concentration of the reinforcing agent and/or the contacting time is/are selected in accordance with a desired mechanical property of the final construct, that is, in accordance with a chemical cross-linking degree that provides a desired mechanical property of the final construct. Typically, for stronger or stiffer constructs, a higher concentration of the reinforcing agent and/or longer contacting times are selected, as long as the viability of cells in the construct is maintained; and for weaker or softer constructs, a lower concentration of the reinforcing agent and/or shorter contacting time are selected. A desired mechanical property of the reinforced construct and/or a corresponding degree of cross-linking can be readily determined by those skilled in the art using available data. The respective contacting time and/or concentration of the reinforcing agent can then be determined either experimentally or by means of predictive calculation tools.

According to some embodiments, the biocompatible small-molecule reinforcing agent is chemically interacted with at least 10, at least 20, at least 30, at least 40, at least 50, %, or more, % of respective chemically compatible groups (e.g., free amine groups as described herein) present in the ECM-based hydrogel (before it chemically interacts with the reinforcing agent). The number of functional groups (which are chemically compatible with the reinforcing agent) in the ECM-based matrix, which chemically interact with the reinforcing agent can be determined by selecting the concentration of the reinforcing agent and/or the contacting time as described herein.

In one embodiment, the reinforcing agent is contacted with the construct once the cells thereof adhere to one another or interact with one another (e.g. via tight junctions, gap junctions).

In another embodiment, the reinforcing agent is contacted with the construct once the construct is at least partially vascularized.

In still another embodiment, the reinforcing agent is contacted with the construct only after at least 50%, 60%, 70%, 80%, 90% of at least one of the cell types thereof express a marker associated with a mature cell type. In still another embodiment, the reinforcing agent is contacted with the construct only after at least 50%, 60%, 70%, 80% or 90% of all of the cell types thereof express a marker associated with a mature cell type.

Preferably, the reinforcing agent is contacted with the construct at least 1 day, 2 days, 3 days, 4 days, 5 days, 6 days, 1 week, 10 days, 2 weeks after seeding/printing. The amount of time may be determined by one of skill in the art according to the cell types used in the construct and the culturing conditions used.

According to another aspect of the present invention there is provided an engineered cellular construct reinforced according to the methods described herein above.

According to another aspect of the invention, there is provided a cellularized engineered construct comprising cells distributed within a chemically cross-linked ECM-based hydrogel, wherein said ECM-based hydrogel is chemically cross-linked by a biocompatible small-molecule reinforcing agent as described herein in any of the respective embodiments and any combination thereof.

According to some of any of the embodiments described herein, the cellularized engineered construct is such that the biocompatible small-molecule reinforcing agent is chemically interacted with (e.g., covalently bound to) at least 10, at least 20, at least 30, at least 40, at least 50, %, or more, % of respective chemically compatible groups (e.g., free amine groups as described herein) present in the ECM-based hydrogel (before it chemically interacted with the reinforcing agent). That is, the number of free chemically compatible groups (e.g., free amine groups) in the ECM-based hydrogel is lower by at least 10%, at least 20%, at least 30%, at least 40%, at least 50%, at least 60%, or more, compared to the same number of free groups in the same ECM-based hydrogel which was not interacted with the reinforcing agent. Determining the number of free functional groups can be performed by analytical measurements known in the art. An exemplary method of determining the number of free amine groups is described in the Examples section that follows, and utilizes spectroscopic measurements (e.g., absorption measurements) upon reacting the construct with a spectroscopically active agent that binds to the respective free groups.

Prior to use, the reinforced constructs may be cultured in suitable media for a length of time to allow for further cell proliferation and growth. The constructs may be cultured for at least one week, 2 weeks, 3 weeks, 4 weeks or even longer. According to a particular embodiment, the constructs are perfused in a perfusion chamber or bioreactor prior to use. According to some embodiments, perfusion is effected subsequent to contacting the construct with the reinforcing agent according to the respective embodiments. Alternatively, perfusion is effected prior to contacting the construct with the reinforcing agent.

In some embodiments, the constructs described herein are used for scientific and/or medical research. Suitable scientific and/or medical research includes both in vivo and in vitro research. In further embodiments, the engineered, constructs described herein, are for in vitro research uses including, by way of non-limiting examples, disease modeling, drug discovery, and drug screening.

The constructs of the present invention are suitable for implantation and may be used for treating any disorder or condition associated with tissue degeneration. In various embodiments, the constructs are suitable for implantation in any vertebrate subject in need of, for example, wound repair, tissue repair, tissue augmentation, tissue replacement, and/or organ replacement. In some embodiments, the constructs are used for wound repair or tissue repair. For example, an engineered sheet is used to temporarily or permanently repair human skin damaged by injury. In some embodiments, the engineered constructs are used for tissue augmentation. For example, an engineered construct is used to temporarily or permanently patch or repair a defect in the muscle wall of a human bladder or stomach. In some embodiments, the engineered tissues are used for tissue replacement. For example, an engineered sheet or tube is used to temporarily or permanently repair or replace the wall of a segment of human small intestine. In some embodiments, the engineered organs are used for organ replacement. For example, an engineered tube is used to temporarily or permanently replace a human fallopian tube damaged by an ectopic pregnancy. In some embodiments, an engineered tubular structure is used to create new connections with organ systems; for example, a smooth muscle-comprising tube could be used to extend a connection from the gastrointestinal system or the kidney through the body wall to enable waste collection in certain disease states. In other embodiments, engineered tubular structures are used to extend the length of certain native tissues (e.g., esophagus, intestine, colon, etc.) to eliminate or ameliorate specific diseases that are congenital in nature (e.g., short gut syndrome, etc.) or occur as a consequence of other diseases or injuries. The engineered, constructs, in various embodiments, are any suitable shape.

In some embodiments, the shape is selected to mimic a particular natural tissue or organ. In some embodiments, the size of engineered constructs, including those bioprinted, change over time. In further embodiments, a bioprinted construct shrinks or contracts after bioprinting due to, for example, cell migration, cell death, cell-adhesion-mediated contraction, or other forms of shrinkage. In other embodiments, a bioprinted tissue or organs grows or expands after bioprinting due to, for example, cell migration, cell growth and proliferation, cell maturation, or other forms of expansion.

In some embodiments, a bioprinted sheet is at least 150 μm thick at the time of bioprinting. In various embodiments, a bioprinted sheet is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 m or more thick, including increments therein. In further various embodiments, a bioprinted sheet is characterized by having a length, width, or both, of about 50, 100, 150, 200, 250, 300, 350, 400, 450, 500, 550, 600, 650, 700, 750, 800, 850, 900, 950, 1000 μm or more, including increments therein. In other various embodiments, a bioprinted sheet is characterized by having a length, width, or both, of about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 mm or more, including increments therein. In other various embodiments, a bioprinted sheet is characterized by having a length, width, or both, of about 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100 cm or more, including increments therein.

In some embodiments, a layer comprising muscle cells or an overall engineered tissue or organ is substantially in the form of a tube or a form that comprises a tube. In further embodiments, a tube is a substantially a rolled sheet or a hollow cylinder. In some embodiments, a bioprinted tube is used to construct an engineered organ. In further embodiments, a bioprinted tube is used to construct an engineered ureter, urinary conduit, fallopian tube, uterus, trachea, bronchus, lymphatic vessel, urethra, intestine, colon, esophagus, or portion thereof. A bioprinted tube has a wide range of suitable dimensions. In some embodiments, the dimensions are selected to facilitate a specific use including, by way of non-limiting examples, wound repair, tissue repair, tissue augmentation, tissue replacement, engineered organ construction, and organ replacement. In further embodiments, the dimensions are selected to facilitate a specific use in a specific subject. For instance, in one embodiment, a tube is bioprinted to repair a particular segment of lymph vessel of a specific human subject. In some embodiments, a bioprinted tube is characterized by having a tubular wall that is at least 150 μm thick at the time of bioprinting. In various embodiments, the wall of a bioprinted tube is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more m thick, including increments therein. In some embodiments, the bioprinted tubes are characterized by having an inner diameter of at least about 250 μm at the time of bioprinting. In various embodiments, the inner diameter of a bioprinted tube is about 50, 60, 70, 80, 90, 100, 125, 150, 175, 200, 225, 250, 275, 300, 325, 350, 375, 400, 425, 450, 475, 500, 600, 700, 800, 900, 1000 μm or more, including increments therein. In other various embodiments, the inner diameter of a bioprinted tube is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 mm or more, including increments therein. In some embodiments, the length of a bioprinted tube is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 mm or more, including increments therein. In other embodiments, the length of a bioprinted tube is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 cm or more, including increments therein.

According to a specific embodiment, the compositions are used for treating a cardiac disorder which is associated with a defective or absent myocardium. The method may be applied to repair cardiac tissue in a human subject having a cardiac disorder so as to thereby treat the disorder. The method can also be applied to repair cardiac tissue susceptible to be associated with future onset or development of a cardiac disorder so as to thereby inhibit such onset or development.

The constructs can be advantageously used to treat disorders associated with, for example, necrotic, apoptotic, damaged, dysfunctional or morphologically abnormal myocardium. Such disorders include, but are not limited to, ischemic heart disease, cardiac infarction, rheumatic heart disease, endocarditis, autoimmune cardiac disease, valvular heart disease, congenital heart disorders, cardiac rhythm disorders, impaired myocardial conductivity and cardiac insufficiency. Since the majority of cardiac diseases involve necrotic, apoptotic, damaged, dysfunctional or morphologically abnormal myocardium, and since the vascularized cardiac tissue of the present invention displays a highly differentiated, highly functional, and proliferating cardiomyocytic phenotype, the method of repairing cardiac tissue of the present invention can be used to treat the majority of instances of cardiac disorders.

According to one embodiment, the constructs described herein can be advantageously used to efficiently reverse, inhibit or prevent cardiac damage caused by ischemia resulting from myocardial infarction.

According to another embodiment, the constructs described herein can be used to treat cardiac disorders characterized by abnormal cardiac rhythm, such as, for example, cardiac arrhythmia.

According to another embodiment, the constructs can be used to treat impaired cardiac function resulting from tissue loss or dysfunction that occur at critical sites in the electrical conduction system of the heart, that may lead to inefficient rhythm initiation or impulse conduction resulting in abnormalities in heart rate.

The method according to this aspect of the present invention is effected by implanting a therapeutically effective amount of the construct of the present invention to the heart of the subject.

As used herein, the term “implantable” refers to being biocompatible and capable of being inserted or grafted into or affixed onto a living organism either temporarily or substantially permanently.

Implantation involves inserting or grafting the construct into a subject. In further embodiments, insertion and/or grafting is performed surgically. In other embodiments, implantation involves affixing a tissue or organ to a subject.

According to one embodiment, implantation of the constructs of the present invention for repair of damaged myocardium is effected following sufficient reduction of inflammation of affected cardiac tissues and prior to formation of excessive scar tissue.

It will be recognized by the skilled practitioner that when administering non-syngeneic cells or tissues to a subject, there is routinely immune rejection of such cells or tissues by the subject. Thus, the method of the present invention may also comprise treating the subject with an immunosuppressive regimen, preferably prior to such administration, so as to inhibit such rejection. Immunosuppressive protocols for inhibiting allogeneic graft rejection, for example via administration of cyclosporin A, immunosuppressive antibodies, and the like are widespread and standard practice in the clinic.

Examples of immunosuppressive agents include, but are not limited to, methotrexate, cyclophosphamide, cyclosporine, cyclosporin A, chloroquine, hydroxychloroquine, sulfasalazine (sulphasalazopyrine), gold salts, D-penicillamine, leflunomide, azathioprine, anakinra, infliximab (REMICADE), etanercept, TNFα blockers, a biological agent that targets an inflammatory cytokine, and Non-Steroidal Anti-Inflammatory Drug (NSAIDs). Examples of NSAIDs include, but are not limited to acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, Cox-2 inhibitors and tramadol.

It is expected that during the life of a patent maturing from this application many relevant printing technologies will be developed and the scope of the term printing is intended to include all such new technologies a priori.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

For any of the embodiments described herein, a reinforcing agent as described herein may be in a form of a salt, for example, a pharmaceutically acceptable salt.

As used herein, the phrase “pharmaceutically acceptable salt” refers to a charged species of the parent compound and its counter-ion, which is typically used to modify the solubility characteristics of the parent compound and/or to reduce any significant irritation to an organism by the parent compound, while not abrogating the biological activity and properties of the administered compound. A pharmaceutically acceptable salt of a compound as described herein can alternatively be formed during the synthesis of the compound, e.g., in the course of isolating the compound from a reaction mixture or re-crystallizing the compound.

In the context of some of the present embodiments, a pharmaceutically acceptable salt of the compounds described herein may optionally be an acid addition salt and/or a base addition salt.

An acid addition salt comprises at least one basic (e.g., amine and/or guanidinyl) group of the compound which is in a positively charged form (e.g., wherein the basic group is protonated), in combination with at least one counter-ion, derived from the selected acid, that forms a pharmaceutically acceptable salt. The acid addition salts of the compounds described herein may therefore be complexes formed between one or more basic groups of the compound and one or more equivalents of an acid.

A base addition salt comprises at least one acidic (e.g., carboxylic acid) group of the compound which is in a negatively charged form (e.g., wherein the acidic group is deprotonated), in combination with at least one counter-ion, derived from the selected base, that forms a pharmaceutically acceptable salt. The base addition salts of the compounds described herein may therefore be complexes formed between one or more acidic groups of the compound and one or more equivalents of a base.

Depending on the stoichiometric proportions between the charged group(s) in the compound and the counter-ion in the salt, the acid additions salts and/or base addition salts can be either mono-addition salts or poly-addition salts.

The phrase “mono-addition salt”, as used herein, refers to a salt in which the stoichiometric ratio between the counter-ion and charged form of the compound is 1:1, such that the addition salt includes one molar equivalent of the counter-ion per one molar equivalent of the compound.

The phrase “poly-addition salt”, as used herein, refers to a salt in which the stoichiometric ratio between the counter-ion and the charged form of the compound is greater than 1:1 and is, for example, 2:1, 3:1, 4:1 and so on, such that the addition salt includes two or more molar equivalents of the counter-ion per one molar equivalent of the compound.

An example, without limitation, of a pharmaceutically acceptable salt would be an ammonium cation or guanidinium cation and an acid addition salt thereof, and/or a carboxylate anion and a base addition salt thereof.

The base addition salts may include a cation counter-ion such as sodium, potassium, ammonium, calcium, magnesium and the like, that forms a pharmaceutically acceptable salt.

The acid addition salts may include a variety of organic and inorganic acids, such as, but not limited to, hydrochloric acid which affords a hydrochloric acid addition salt, hydrobromic acid which affords a hydrobromic acid addition salt, acetic acid which affords an acetic acid addition salt, ascorbic acid which affords an ascorbic acid addition salt, benzenesulfonic acid which affords a besylate addition salt, camphorsulfonic acid which affords a camphorsulfonic acid addition salt, citric acid which affords a citric acid addition salt, maleic acid which affords a maleic acid addition salt, malic acid which affords a malic acid addition salt, methanesulfonic acid which affords a methanesulfonic acid (mesylate) addition salt, naphthalenesulfonic acid which affords a naphthalenesulfonic acid addition salt, oxalic acid which affords an oxalic acid addition salt, phosphoric acid which affords a phosphoric acid addition salt, toluenesulfonic acid which affords a p-toluenesulfonic acid addition salt, succinic acid which affords a succinic acid addition salt, sulfuric acid which affords a sulfuric acid addition salt, tartaric acid which affords a tartaric acid addition salt and trifluoroacetic acid which affords a trifluoroacetic acid addition salt. Each of these acid addition salts can be either a mono-addition salt or a poly-addition salt, as these terms are defined herein.

Further, a reinforcing agent as described herein, including the salts thereof, can be in a form of a solvate or a hydrate thereof.

The term “solvate” refers to a complex of variable stoichiometry (e.g., di-, tri-, tetra-, penta-, hexa-, and so on), which is formed by a solute (the heterocyclic compounds described herein) and a solvent, whereby the solvent does not interfere with the biological activity of the solute.

The term “hydrate” refers to a solvate, as defined hereinabove, where the solvent is water.

The reinforcing agent described herein can be used as polymorphs and the present embodiments further encompass any isomorph of the compounds and any combination thereof.

The reinforcing agent described herein encompass any stereoisomer, including enantiomers and diastereomers, of the compounds described herein, unless a particular stereoisomer is specifically indicated.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an (R) or an (S) configuration and any combination, and compounds according to some embodiments of the present invention, can have any their chiral centers exhibit an (R) or an (S) configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Materials and Experimental Methods

### Synthesis of SOx:

Oxidized sucrose (SOx) was prepared following a previously established protocol [Liu et al, Macromol. Mater. Eng. 2015, 300, 414]. Briefly, a 33 mm solution of D-sucrose (Bio-Labs) in acetate buffer (pH=5) was stirred at room temperature while a 0.2 M NaIO₄ (Fischer Chemical) solution was added dropwise at the rate of 1 mL min⁻¹ by a syringe pump (NE-1000; New Era Pump Systems). The entire solution was kept in the dark and allowed to stir overnight. A 1M BaCl₂ (Acros Organics) solution was added dropwise until further addition of the BaCl₂ resulted in no further precipitation of iodine salts. The mixture was thereafter filtered to remove precipitates, and a 0.1 M NaOH solution was carefully added until the solution reached a pH of about 7.4. The mixture was again filtered to remove the barium salts, then lyophilized and stored at −20° C. until use.

The compound's structure was verified by ¹H-NMR (data not shown). The appearance of the peak at δ=8.5 is indicative of the aldehydes that were successfully synthesized.

### Preparation of Decellularized Omentum Hydrogel:

Porcine omental tissue was obtained and decellularized as previously described [Edri et al, Adv. Mater. 2019, 31, 1970007]. Briefly, the omental tissues (Kibbutz Lahav, Israel) were washed with phosphate-buffered saline (PBS), large blood vessels were excised from the tissue, and the tissue was transferred to a hypotonic buffer (10 mm trisaminomethane (Hy Laboratories), 5 mm ethylenediaminetetraacetic acid (Sigma-Aldrich), 1 μm phenylmethanesulfonylfluoride (Sigma-Aldrich), at pH=8). After 1 hour at room temperature, the tissue underwent three cycles of freeze-thawing (−80° C.) in the hypotonic buffer. The tissue was then washed for 30 minutes each with 70% (v/v) ethanol (Bio-Lab), 96% (v/v) denatured ethanol (Bio-Lab), and three times with acetone (Bio-Lab). Next, the tissue was soaked for 24 hours in a 40% (v/v) solution of acetone in n-hexane (Bio-Lab) with three solution changes. The next day, the tissue was washed for 30 minutes with 96% ethanol, and was thereafter incubated overnight at 4° C. in 70% ethanol. The tissue was then washed four times with PBS and incubated overnight in a 0.25% solution of trypsin and EDTA (Biological Industries), followed by four more washings with PBS and incubation for 24 hours in a 1.5 m NaCl (Bio-Lab) solution with three solution changes. The tissue was then washed in a solution of 50 mm trisaminomethane (pH=8) and 1% Triton-X100 (Sigma-Aldrich) for 1 hour. Finally, the now decellularized extracellular matrix (dECM) was washed thoroughly with PBS and double distilled water (DDW) and frozen (−20° C.).

The dECM was dried in a lyophilizer, then ground into flakes with a Wiley Mini-Mill (Thomas Scientific) and dissolved in a 0.1 M HCl solution to a level of 1.11% (w/v). The dECM was then processed enzymatically via the addition of 1 mg porcine pepsin (Sigma-Aldrich) per 10 mg dECM, which was left stirring at room temperature until no large collagen chunks were discernable (3-4 days). Following enzymatic digestion, the solution's pH was adjusted to 7.4 by the addition of 5M NaOH. Dried Dulbecco's modified Eagle medium (DMEM/F12) (Biological Industries) was added at a 10× concentration in DDW to reach a final working concentration of 1×. This also reduced the final concentration of dECM to 1% (w/v). The entire solution was filtered through a sterile, 70 μm, nylon cell filter (Bar Naor) before use, and 0.1% Pen/Strep (Biological Industries) was added.

Collagen Dissociation with SDS:

Hydrogel samples were submerged in a fresh 1% solution of Sodium dodecyl sulfate (Sigma) in PBS. The absorbance of light of wavelength 600 nm (Infinite M200 Pro; Tecan, Switzerland) was assessed every 15 minutes at several different locations within the well. The highest absorbance was taken as the reading.

Collagen Degradation with Collagenase:

Hydrogel samples were immersed in a solution of 1 U mL⁻¹ Collagenase Type II (Worthington, Lakewood, NJ, USA). The absorbance of light of wavelength 600 nm was assessed daily at several different locations within the well. The highest absorbance was taken as the reading. The collagenase solution was refreshed every 48 hours.

iPSC Culture:

iPSCs were generated from omental stromal cells. Documentation regarding the cell line can be found at www(dot)hpscreg(dot)eu/cellline/BGUi013-A). The undifferentiated cells were cultivated on 10-cm culture plates pre-coated with Matrigel (BD, Franklin Lakes, NJ, USA) diluted to 250 g mL⁻¹ in DMEM/F12 (Biological Industries). Cells were maintained in NutriStem (Biological Industries) medium containing 0.1% penicillin/streptomycin (Biological Industries) and cultured under a humidified atmosphere at 37° C. with 5% CO₂. Medium was refreshed daily, and cells were passaged at 80% confluence by treatment with ReLeSR (Stemcell Technologies, Vancouver, Canada).

CM Differentiation from iPSCs:

Prior to differentiation, cells were dissociated with Accutase (StemCell Technologies) and passaged to 6-well plates coated with Matrigel as before. NutriStem (Biological Industries) was refreshed daily until iPSCs reached 100% confluence. At that point (Day 0), medium was changed to RPMI (3 mL) (Biological Industries), supplemented with 0.5% L-glutamine (Biological Industries), B27-Insulin (Invitrogen, Carlsbad, CA, USA), and 4.5 μm CHIR-99021 (Tocris, Bristol, UK). On Day 2, the medium was changed to RPMI (3 mL) supplemented with 0.5% L-glutamine, B27-Insulin, and 5 μm IWP-2 (Tocris). On Day 4, the medium was changed to RPMI (3 mL) supplemented with 0.5% L-glutamine and B27-Insulin, and this medium was refreshed on Day 6. On Day 8 the medium was changed to RPMI (3 mL) supplemented with 0.5% L-glutamine and B27, and this medium was refreshed on Day 10. From Day 12, medium was changed to M-199 (Biological Industries), supplemented with 0.1% penicillin/streptomycin, 5% fetal bovine serum (FBS, Biological Industries), 0.6 mM CuSO₄, 0.5 mM ZnSO₄, and 1.5 mM vitamin B12 (Sigma-Aldrich). This medium was refreshed every other day.

### EC Culture:

Primary human umbilical vein endothelial cells (HUVECs) were purchased commercially (Angio-Proteomie), and were maintained in Endothelial Growth Medium (EGM-2) (Lonza) supplemented with an additional 1.5% (v/v) FBS. The medium was refreshed every other day.

### Mathematical Modeling:

Finite element analysis was computed using commercially available software. Mass transport simulations were run using the following parameters:
Diffusion coefficient of oxygen [Mattei et al, Processes 2014, 2, 548]: (1×10⁻³ mm² s⁻¹);
Maximum cellular oxygen consumption rate [Brown et al, Biotechnol. Bioeng. 2007, 97, 962]: (5.44×10⁻⁸ nmol cell⁻¹ s⁻¹);
Michaelis-Menten constant for oxygen consumption: 3.49 nmol mL⁻¹ [Brown et al, 2007, supra];
Critical oxygen concentration to account for necrosis [Mattei et al, 2014, supra]: 2.64×10⁻³ mol mL⁻¹);
Initial oxygen concentration [Mattei et al, 2014, supra]: 0.2 μmol mL⁻¹) and Cell density (100×10⁶ cells mL⁻¹) [Noor et al, Adv. Sci. 2019, 6, 1900344].

### Preparation of Granula:

Granula was prepared according to previously reported protocols [Pesce et al, Nat. Rev. Cardiol. 2022, 20, 309]. Briefly, a solution of sodium alginate, xanthan gum, and sodium chloride was prepared with uniformly distributed calcium carbonate. To this solution, gluconic acid δ-lactone was added, the solution was mixed thoroughly, and the entire mixture was allowed to sit overnight. After 24 hours, the mixture was dissolved in DDW and the entire contents were homogenized. The homogenized stock solution was then set aside at 4° C.

Prior to use, the stock solution was centrifuged at 15,800 g for 20 minutes and the supernatant was removed. The pellet was washed three times by re-suspension in DMEM (Biological Industries) with an addition of 20 mm HEPES (Thermo-Fisher). The final pellet formed the working granula.

### Preparation of Gelatin Microparticles:

Gelatin microparticles were prepared using an adapted protocol [Duhoranimana et al, Food Hydrocolloids 2017, 69, 111]. A solution of 0.9% Gelatin Type B 225 Bloom (Sigma-Aldrich) with 0.1% carboxymethylcellulose sodium salt (Sigma-Aldrich) in DDW was heated to 60° C. while stirring. After 2 hours, the temperature was lowered to 45° C. A solution of 1% acetic acid was added dropwise with stirring until reaching the clouding point, and the solution was then allowed to continue stirring for 15 minutes. The solution was then placed in an ice bath and stirring continued for another 15 minutes. An excess of acetone was then added, and the solution was stirred for 15 more minutes. The solution was then centrifuged at 4° C. and 3000 g for 15 minutes. The pellet was washed via resuspension in PBS and another centrifugation as before. The wash was repeated, and the final pellet was kept at 4° C. until its use.

### Printing of Cardiac Patches:

iPSC-derived CMs grown on Matrigel coated plates were incubated for 10 minutes with TrypLE Express (Gibco, Waltham, MA, USA). Colonies were then mechanically triturated, and cells were centrifuged at 300 g for 5 minutes. The supernatant was removed, and ECM-based hydrogel was added at a ratio of 1 mL per 200 million cells.

ECs were incubated with a 0.25% solution of trypsin and EDTA (Biological Industries) for 5 minutes to dissociate cells. The cells were collected in an excess of DMEM and centrifuged at 300 g for 5 minutes. The supernatant was removed and the cells were re-suspended in the gelatin microparticles at a ratio of 15M cells per 1 mL of gelatin microparticles.

Both bioinks were printed using a high-precision print head on a 3DDiscovery printer (regenHU, Villaz-St-Pierre, Switzerland). The CM-ink was printed through a 25G needle. The EC-ink was printed through a 30G needle. The supporting granula was printed through a 20G needle.

Following printing, patches were placed in a humidified incubator (37° C., 5% CO₂) for 25 minutes, during which time the omentum patches underwent a process of physical cross-linking. After 25 minutes, M-199 (as described above) with all the additions of the EGM-2 bullet kit (Lonza) was added. On Day 2, this medium was refreshed. The next day, the medium was changed to M-199 with all the additions of the EGM-2 bullet kit (Lonza) plus 0.03% SOx. After overnight incubation, the patches' medium was changed back to what it had been on Day 0, and the medium was thereafter changed every 2-3 days.

### Compressive Modulus:

The compressive modulus measurements were performed using a Discovery HR-3 Hybrid Rheometer (TA Instruments, DE) with a 20 mm diameter parallel plate geometry and a Peltier plate to maintain the sample temperature. Bulk modulus tests were performed by compressing the samples at a fixed rate of 100 μm s⁻¹.

### Perfusion:

For the perfusion experiments, samples were printed directly into a custom plastic chamber, which was designed using open source computer-aided design software and printed with a Max X DLP 3D printer (Asiga; Sydney, Australia). The walls of the chamber contained a small hole matching the diameter of a 27G needle that lined up precisely with the printed lumen, so that a needle could be easily inserted directly into the blood vessel. To image the perfusion, a peristaltic pump was connected to the chamber and rhodamine-dextran (MW of about 10 kDa, Sigma #R8881) was pumped through the lumens. To perform perfusion following injection, the samples were removed from the custom chamber and injected through a syringe. Because it was not possible to obtain the precision necessary to use the chambers following injection, 27G needles were manually inserted into the approximate location of the lumen by a process of trial and error.

### Calcium Imaging:

Samples were incubated with 10 μm Fluo-4 AM (Invitrogen) and 0.1% Pluronic F-127 (Sigma-Aldrich) in Tyrode's solution (1.8 mm CaCl₂, 5 mm glucose, 10 mm HEPES, 1 mm MgCl₂, 5.4 mm KCl, 135 mm NaCl, 0.33 mm Na₃PO₄) for 20 minutes at 37° C. Samples were then imaged using a binocular microscope (SMZ18, Nikon). Movies were acquired using an ORCA-Flash 4.0 digital complementary metal-oxide-semiconductor (CMOS) camera (Hamamatsu Photonics) at a rate of 100 frames s⁻¹.

For the spatiotemporal heat maps, movies of the calcium signals were filtered using ImageJ (FIJI) and analyzed using MATLAB software (MathWorks, MA, USA), where a custom script was employed for detecting the time-point of maximum change in intensity for every pixel.

### Antibody List:

Antibodies for stem cells: Goat to Oct4 (ab27985), 1:100, Abcam.

Rabbit to Ki67 (ab16667), 1:250, Abcam.

Antibodies for cardiac cells: Rabbit to Sarcomeric Alpha Actinin (ab68167), 1:200, Abcam.

Mouse to Sarcomeric Alpha Actinin (ab9465), 1:200, Abcam.

Antibodies for endothelial cells: Mouse to CD31/PECAM-1 (P8590), 1:250, Sigma.

Secondary antibodies: Donkey Anti-Rabbit (Dy-Light 488), 1:250, Bethyl Laboratories. Goat Anti-Rabbit (Alexa Fluor 488) (ab2338046), 1:250, Jackson ImmunoResearch. Goat Anti-Mouse (Alexa Fluor 555) (ab150118), 1:500, Abcam. Goat Anti-Mouse (Alexa Fluor 647) (ab2338902), 1:250, Jackson ImmunoResearch. Donkey Anti-Goat (Alexa Fluor 647) (ab150135), 1:500, Abcam.

For detection of nuclei, cells were incubated with Hoechst 33258, 1:100, Sigma. Cell

Proliferation Staining: Deep Red (ab176736), Abcam. Green (ab176735), Abcam.

### Statistical Analysis:

Statistical analyses were presented as mean±standard deviation on the basis of at least three replicates. Differences between samples were assessed by the relevant tests (details for each experiment were provided alongside the data), and p<0.05 was considered significant. Analyses were performed using GraphPad Prism 8 (Version 8.4.2) for Windows (GraphPad Software).

### Assessment of Amine Content:

Amine content was quantified by the stoichiometric reaction of amines with ninhydrin following a simplified protocol [Zhang et al, Anal. Biochem. 2013, 437, 46]. Aliquots of hydrogel (1 mL) were taken and either reacted with SOx or used as a control. Samples were lyophilized and then immersed in a 0.05% acetic acid solution (1 mL). To this was added 2% Ninhydrin in ethanol (w/v) (1 mL). The mixture was boiled for 90 minutes. Aliquots were taken and the absorbance of the solution was recorded at 570 nm (Infinite M200 Pro; Tecan, Switzerland).

### SEM Images:

Hydrogel samples were fixed with 2.5% glutaraldehyde (24 hours), followed by a series of washes with ethanol-water solutions with an increasing percentage of ethanol (from 35% to 100% (v/v)). All samples were dried using critical point drying (Balzers), sputter-coated with gold (Polaron E 5100, Quorum Technologies, Lewis, UK) and observed using a Gemini 300 HRSEM (Zeiss, Germany).

### NMR Spectra:

NMR spectra were obtained from an Avance III NMR, 11.7 Tesla (500.16 MHz for 1H) (Bruker, Massachusetts, US). Oxidized sucrose was dissolved in D₂O for the recordings.

### Rheological Testing:

Rheological measurements were performed using a Discovery HR-3 Hybrid Rheometer (TA Instruments, DE) with a 20 mm diameter parallel plate geometry and a Peltier plate to maintain the sample temperature. Shear thinning experiments were performed by varying the strain rate while maintaining a fixed strain of 20%.

FACS Assay of iPSCs:

Cells were dissociated with Accutase™ (StemCell Technologies), centrifuged at 300 g, and resuspended in Flow Cytometry Staining Buffer (R&D Systems). Cells were aliquoted and compared against a control aliquot and an aliquot stained with a control isotype. The antibodies used were (Miltenyi Biotech): TRA-1-60 (REA157), SSEA-1 (REA321), SSEA-4 (REA101), Control antibody (REA293). Data was collected on a CytoFLEX S Flow Cytometer (Beckman Coulter) and analyzed using their CytExpert software.

FACS Assay of iPSC-CMs:

Cells were dissociated with TrypLE™ Express (Gibco, Waltham, Massachusetts), centrifuged at 300 g, and resuspended in eBioscience™ Permeabilization Buffer and Fixation/Perm Diluent Buffer (Invitrogen). Cells were subsequently blocked with Bio-Pure Human Serum Albumin 10% solution (Biological Industries) diluted to 0.1% in PBS.

The cells were aliquoted for controls and isotype and stained for (Miltenyi Biotech): Cardiac Troponin (REA400) and REA Control (REA293). Data was collected on a CytoFLEX S Flow Cytometer (Beckman Coulter) and analyzed using their CytExpert software.

### Cell Viability:

Primary human umbilical vein endothelial cells (HUVECs) (Angio-Proteomie) were maintained in Endothelial Growth Medium (EGM-2) (Lonza) supplemented with an additional 1.5% (v/v) fetal bovine serum (FBS) (Biological Industries). Daily, PrestoBlue™ Cell Viability Reagent (Invitrogen) was added and cells were incubated for a fixed time interval. The medium was then transferred to a clean, opaque tissue culture well plate and fresh medium was added to the cells. The fluorescence was measured at 560/590 nm (Infinite M200 Pro).

### Histological Staining:

Cells or tissues were fixed in 4% formaldehyde, permeabilized with 0.1% (v/v) Triton X-100 (Sigma-Aldrich), blocked with PBS containing 1% bovine serum albumin (BSA) and 10% FBS, and stained with primary antibodies followed by secondary antibodies (as indicated in the antibody list). The samples were imaged using an upright confocal microscope (Nikon Eclipse NI-E) and inverted fluorescence microscope (Nikon Eclipse TI-E). Images were processed and analyzed using the NIS elements software (Nikon Instruments). Representative images from at least three different biological experiments were chosen.

### Example 1

### Design

Ideally, a method for tissue reinforcement would start from natural, ECM-based polymers in which cells are encapsulated before tissue fabrication. Following fabrication, the reinforcement protocol would be applied uniformly to the entire structure to improve the mechanical properties of the tissue. Additionally, it would be advantageous if the reinforcement protocol could be delayed, as modifying or hardening the ECM while the cells are still self-arranging may hinder the ability of the cells to move, spread, and interact with each other.

In a search for methodologies for implementing the above recognition, the present inventors have designed and successfully practiced a method for enhancing the robustness of (reinforcing) an engineered cardiac patch post-tissue assembly. The designed methodology is illustrated in FIG. 1A, and is based on a two-step system. First, the aforementioned physical gelation of the ECM-based hydrogel is exploited to create a soft environment for the initial phases of tissue maturation. Then, a small reinforcing biomolecule in growth media is incorporated after tissue assembly and during its maturation process, such that it is allowed allows to penetrate deep into the entire engineered structure and significantly and safely increase the tissue's strength from within.

As schematically illustrated in FIG. 1A, cardiac and endothelial cells were combined with hydrogels to create bioinks, which were 3D printed to form a natively vascularized tissue. After allowing the cells to self-organize in a soft gel, a tissue-penetrating small molecule was introduced to homogenously reinforce the tissue. Ultimately, the enhancement in the tissue's mechanical properties enabled it to be subjected to strong compression and shear stress, and even injected, without harming it or changing its physiological properties.

As is described in further detail in the following examples, it has been demonstrated that tissue manipulation, including folding, compressing, and injecting a reinforced tissue to simulate the stresses that would be applied in a minimally invasive operation used to introduce a cardiac patch into a damaged heart, caused no damage to the nano-, micro-, or macroscale structures of the engineered tissue. Most importantly, tissue function was not affected.

As an exemplary tissue-penetrating small molecule reinforcing agent, an oxidized form of sucrose (SOx), shown in FIG. 1B, has been selected. This molecule has been previously shown to have a reduced toxicity profile when used to cross-link non-cellularized scaffolds. Thus, by crosslinking scaffolds and then adding cells, residual SOx molecules left in the scaffold do not negatively impact cell viability [Kamimura et al, J. Biomed. Mater. Res, Part A 2014, 102, 4309; Jalaja, N. R. James, Int. J. Biol. Macromol. 2015, 73, 270; Liu et al, Macromol. Mater. Eng. 2015, 300, 414].

Therefore, SOx was chosen as a non-limiting small, fast-diffusing molecule to reinforce a pre-printed structure The present inventors have hypothesized that adding SOx to the tissue growth medium as a post-printing and post-tissue assembly reinforcing agent would give the cells time to form cell-cell and cell-matrix interactions within the soft hydrogel, as schematically illustrated in FIG. 2A.

SOx was synthesized in accordance with previous protocols (see, the Materials and Methods section hereinabove), and its ability to diffuse into an acellular, ECM-based hydrogel construct to provide mechanical reinforcement was assessed. SOx is a poly-aldehyde that reacts with amine moieties present in the native ECM (e.g., collagen) via a Schiff base “click” reaction, to form imine bridges, as schematically illustrated in FIG. 2B.

As all previous studies with SOx have applied the molecule to non-cellularized scaffolds to which cells were later added, it was assessed whether SOx could be used to reinforce fully cellularized tissues without causing cell death.

Cells were incubated for 48 hours in 2D culture in order to allow toxic effects to become discernable. Data are presented in FIG. 2C as mean±standard deviation (SD), n=4. All values were normalized to the average value of the control group. P-values were calculated using a two-tailed, unpaired, parametric t-test assuming that all samples have the same distribution.

Cell viability testing revealed that the presence of SOx had no impact on cell viability at concentrations lower than 0.1% in the culturing medium, as shown in FIG. 2C, and this concentration served as the upper threshold in further studies.

### Example 2

### Reinforcement Effect on ECM-Based Hydrogels

The mechanical properties of an exemplary native ECM-based hydrogel and of cross-linked hydrogels were assessed by rheological measurements. Shear thinning Measurements were conducted at 37° C. Data are presented as mean±SD, n=3. P-values were calculated using a Comparison of Fits test with an Extra Sum of Squares F-test.

The data are presented in FIG. 2D and show that at all frequencies, the reinforced hydrogels demonstrated superior mechanical properties. While a large increase in gel viscosity was observed between the control gel and a sample cross-linked with 0.03% SOx, a smaller increase was seen when increasing the SOx concentration to 0.07%. Therefore, 0.03% SOx was used in further studies.

Alongside the rheological testing, the bulk mechanical properties of the hydrogel were also assessed. Samples were compressed, and the linear elastic modulus was calculated. Data are presented as mean±SD, n=3. P-values were calculated using a one-tailed, unpaired, parametric t-test assuming that all samples have the same distribution.

The data are presented in FIG. 2E, revealing significant reinforcement of the hydrogel.

To determine the ability of SOx to react with an ECM-based hydrogel, the concentration of amine groups present in the ECM before and after exposure to the Sox was assessed. The data are presented in FIG. 2F. As can be seen, the absorbance of a solution of ninhydrin that was allowed to react with the hydrogel was normalized to the control value and showed that 46% of the amines had reacted with the Sox cross-linker. Data are presented as mean±SD, n=4. All values were normalized to the average value of the control group. P-values were calculated using a one-tailed, unpaired, parametric t-test assuming that all samples have the same distribution.

The morphology of ECM-based hydrogel before and after exposure to Sox was tested and the obtained data is shown is FIGS. 2G-I.

High-resolution imaging of non-crosslinked ECM hydrogel (before exposure to Sox) and of cross-linked ECM hydrogel (after exposure to Sox) are presented in FIGS. 2G and 2H, respectively (Scale bar=1 micrometer), and revealed that the reinforcement with SOx proceeded without altering the internal morphology.

In FIG. 2I the data shown in the obtained images was used to evaluate the pore size, and further show that the reinforcement protocol did not alter the overall mesh formation of the hydrogel. The size of the holes in the mesh were calculated before and after crosslinking, and no difference was observed between the samples. Data in FIG. 2I is presented as mean±SD, n=3, and each image had at least 180 pores whose area was determined using ImageJ. P-values were calculated using a two-tailed, unpaired, parametric t-test assuming that all samples have the same distribution.)

These results suggest that the SOx molecules do not form new adhesion points between the hydrogel fibers, but rather chemically cross-link the existing entanglement points that had already been cross-linked physically. Additionally, the fact that the fibers did not compact during reinforcement suggests that SOx is able to penetrate deep into the tissue and provide uniform reinforcement. It is to be noted that this stands in contrast to most chemical cross-linkers, which cause the polymer network to become more tightly packed, slowing the diffusion rate into the gel and leading to non-uniform cross-linking [see, for example, Wu et al, J. Phys. Chem. B 2009, 113, 3512].

Because cells are uniformly distributed throughout the entire structure, this represents a significant advantage of using SOx as a reinforcer.

To further support these findings, both the control and reinforced hydrogels were exposed to sodium dodecyl sulfate (SDS). SDS is a detergent that binds to peptide chains, imparts a negative charge, and causes them to repel one another [Lambin and Rochu, J. M. Fine, Anal. Biochem. 1976, 74, 567; Reynolds and Tanford, J. Biol. Chem. 1970, 245, 5161].

Because the addition of SDS leads to electrostatic repulsion, weak, physically entangled gels disintegrate in its presence while more strongly, chemically cross-linked gels swell but do not break.

The data obtained for hydrogel disintegration in SDS is shown in FIG. 2J. Data presented as mean±SD, n=3. All values normalized to the average reading across all six samples at t=0. P-values were calculated using a two-tailed, unpaired, parametric t-test assuming that all samples have the same distribution). As compared to the pristine non-reinforced hydrogel, which rapidly disintegrated overnight, the reinforced hydrogel remained intact and only swelled due to the chemical cross-linking that had occurred.

Degradation studies were also performed using the biologically relevant enzyme collagenase, and the obtained data is shown in FIG. 2K. Data presented as mean±SD, n=3. Each sample was individually normalized to its initial value. P-values were calculated using a two-tailed, unpaired, parametric t-test assuming that all samples have the same distribution.

As shown in FIG. 2K, when non-reinforced hydrogels were exposed to collagenase, 61% rapidly degraded, with only 39% of the gel remaining after two weeks. After reinforcement, however, there was no significant degradation within this time period.

Both of the degradation studies therefore support the observation that SOx provides a uniform reinforcement throughout the hydrogel and does not merely create a cross-linked outer shell.

### Example 3

### Tissue Manufacturing

For the bioprinting of engineered, human vascularized cardiac tissue, two mature cell types were employed: cardiomyocytes (CM) for the parenchymal tissue and endothelial cells (EC) for the blood vessels therewithin. As cardiac cells are terminally differentiated cells that cannot proliferate under normal conditions, human induced pluripotent stem cells (iPSCs) were conceived as an expandable cell source.

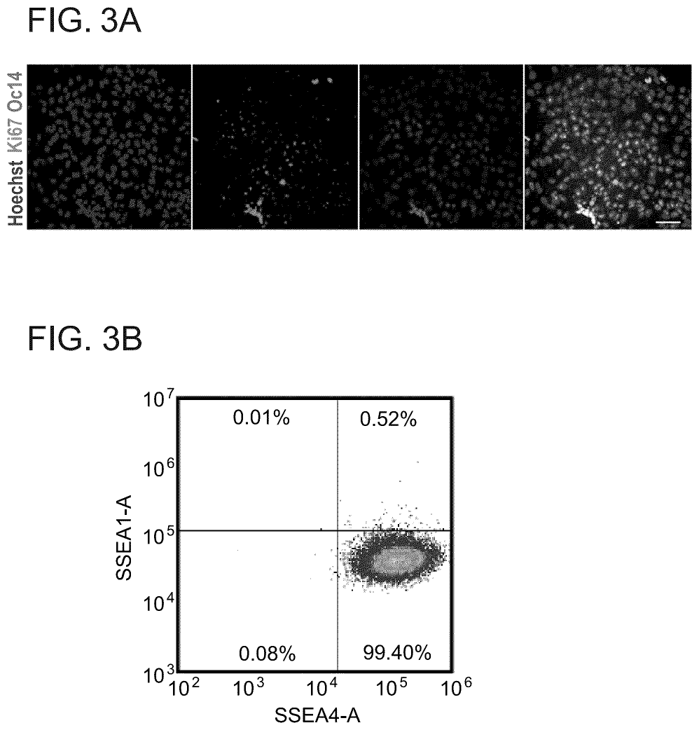

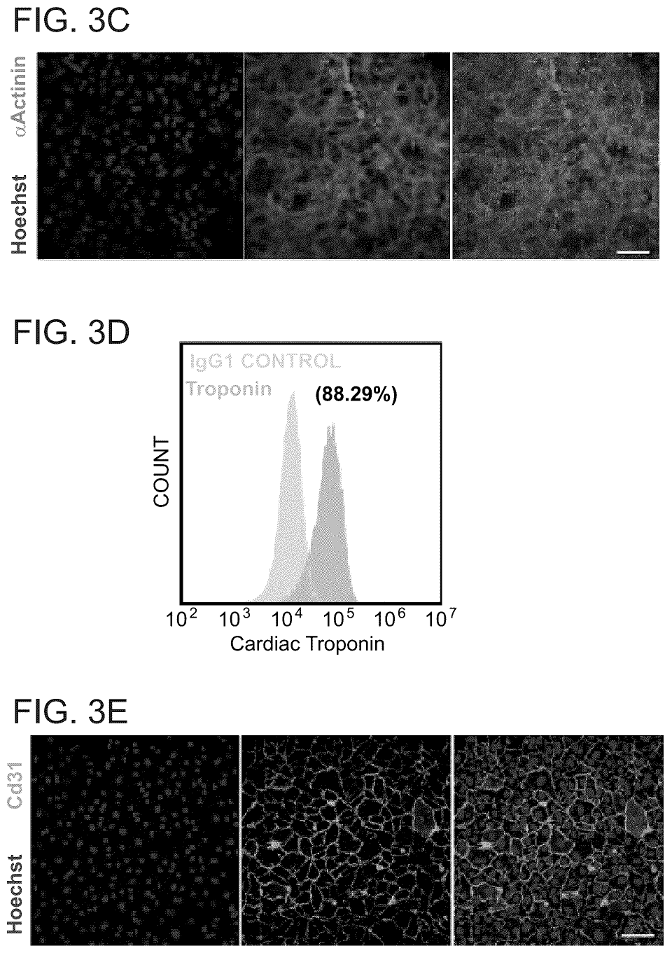

iPSCs were carefully maintained, and their pluripotency was regularly assessed. As shown in FIG. 3A, immunostaining showed a high degree of expression of Oct4, a nuclear pluripotency marker, and Ki67, a nuclear marker of proliferation [Edri et al, 2019, supra; Wertheim et al, Adv. Sci. 2022, 9, 2105694. As shown in FIG. 3B, flow cytometry confirmed the expression of SSEA-4, a membrane-based protein indicative

of pluripotency, and the lack of expression of SSEA-1, an embryonic marker that is notably absent from human iPSCs [Riordon, K. R. Boheler, in The Surfaceome: Methods and Protocols, (Eds.: K. R. Boheler, R. L. Gundry) Springer, New York 2018, p. 127].

Next, the iPSCs were differentiated into cardiomyocytes (iPSCCMs) using previously published protocols [Edri et al, 2019, supra]. FIG. 3C presents images obtained in immunostaining assay as described herein (Scale bar: a=50 μm), and show that efficient differentiation to CMs was assessed for cardiac sarcomeric actinin. Differentiated cardiomyocytes expressed cardiac sarcomeric actinin to a large extent. The cells were elongated with striated sarcomeres.

FIG. 3D presents data obtained for as representative sample in flow cytometry analysis of iPSC-CMs, which revealed that 88.29% of the cells were positive for cardiac troponin. Additionally, the functionality of the iPSC-CMs was assessed by calcium imaging (data not shown).

For the formation of blood vessels, primary human umbilical vein endothelial cells (HUVECs) were selected as a model cell type. The endothelial cells (ECs) were stained for CD31, an endothelial cell adhesion molecule, and the immunostaining images are presented in FIG. 3E (Scale bar=100 μm). The two fluorescent channels are separated into their individual components (Left and middle panels) and superimposed (right panel).

In order to design a cardiac patch that is clinically relevant, the patch should preferably incorporate a high level of vascularization [Rouwkema and Khademhosseini, Trends Biotechnol. 2016, 34, 733]. Potential vascular architectures were assessed via finite element analysis to ensure that the printed blood vessels would be able to provide sufficient oxygen to all of the cells in the tissue.

Modeling parameters were based on publically available literature data [Noor et al, 2019, supra; Brown et al, Biotechnol. Bioeng. 2007, 97, 962; Mattei et al, Processes 2014, 2, 548]. The model included two assumptions intended to overcorrect and ensure its effectiveness. First, the model treats the patch as though it were implanted in vivo, such that there is no surrounding growth medium from which oxygen can diffuse into the tissue. Secondly, the rate of the cellular consumption of oxygen was taken to be the maximal value for cardiomyocyte metabolism.

Blood vessels were added manually, one at a time, until the entire core of the patch was oxygenated to at least 2.64×10⁻³ mmol L⁻¹, the threshold below which a tissue becomes necrotic, as schematically presented in FIG. 4A (Scale bar=3 mm) [Mattei et al, 2014, supra].

Thereafter, the designed cardiac tissue was printed, as shown in the photographs presented in FIG. 4B (Scale bar=3 mm). The cardiac tissue was constructed by encapsulating iPSC-CMs in the ECM-hydrogel and extruding the cell-laden bioink layer-by-layer. In order for the tissue to maintain its structural integrity during printing two types of temporary supporting materials were used. Externally, a non-cellularized supporting hydrogel based on alginate and xanthan gum (“granula”) was used, and internally gelatin was printed as a sacrificial material [Shapira et al, Biomed. Mater. 2020, 15, 045018]. The lumen of the blood vessels was created by printing ECs in a gelatin slurry. The microparticles slurry was produced so that the gelatin could be printed below room temperature to match the conditions needed for printing the ECM bioink. As can be seen in FIG. 4B, the support material (yellow) provided external stabilization, and a cardiomyocyte-laden ink (represented in red) and endothelial cell-laden ink (represented in blue) were deposited layer-by-layer.

By utilizing 3D-printing technology, distinct cell types could be localized to their appropriate positions. The cells were marked with a fluorescent cytopainter before printing, and immediately following printing, the overall localization of cells was assessed to ensure that CMs filled the parenchyma while the endothelial cells were localized to the blood vessels, as demonstrated in FIG. 4C (Scale bar=3 mm).

When incubated at 37° C., the ECM hydrogel physically cross-linked while the gelatin liquefied and flowed out of the patch, leaving behind the vascular tree and the ECs that adhered to the ECM lining the lumens.

Printed patches were matured for a week. For the first three days after the fabrication of the tissue, the cells were allowed to mature within the soft, non-reinforced, physically cross-linked gel. This environment matches the soft environment of the fetal heart in its early stages [Pesce et al, Nat. Rev. Cardiol. 2022, 20, 309; Majkut et al, Curr. Biol. 2013, 23, 2434]. After several days, the tissues began to spontaneously contract, indicating that the iPSC-CMs had reached a basic level of maturation and organization.

Once the cardiac tissue was printed, and cells were allowed to mature and organize, the tissues' robustness was reinforced by the incorporation of SOx into the growth medium. The structure and functionality were then assessed at nano-, macro-, and cellular levels.

The tissue was perfused to ensure the integrity of the macrostructure. By using fluorescently labeled endothelial cells, a dye flowing through the cell-lined lumens can be observed, as can been seen in FIG. 4D (Scale bar=3 mm). The fully connected and hollow blood vessels allowed the tissue to be fully perfused through the cell-lined blood vessels.

At the cellular level, cardiac cell morphology was examined within the printed tissue by immunostaining, and the obtained image is presented in FIG. 4E (Scale bar=20 micrometer). CMs were imaged to ensure the maturation, elongation, and alignment of the sarcomeres. The cardiomyocytes elongated and aligned within the 3D tissue.

The functionality of the tissue was also assessed. Calcium transients were measured in the contracting tissue, and individual pulses were followed across the entirety of the patch to ensure a complete network of CMs. The obtained data is presented in FIG. 4F (Scale bar=1 mm), as follows: (I) distinct points, represented as colored circles, were selected from the overall image; (II) the fluorescent signal was tracked as a function of time at each point, and the derivative of the plot was taken as a function of time; (III) a single action potential was isolated and tracked as it moved across the indicated points, showing a clear progression of the signal; (IV) an individual pulse plotted as it moves across the tissue. The synchronous contraction indicated a high level of intercellular communication and tissue organization.

### Example 4

### Reinforcement Measurements

The engineered cardiac tissues (d=3.6 cm, h=2.5 mm) were folded and compressed, placed inside a syringe (d internal=0.9 cm), and injected out the other end to simulate the conditions applied in a minimally invasive procedure, as shown in the photographs presented in FIG. 5A (Scale bar=1 cm), as follows: (I) a photograph of an acellular hydrogel (d=3.6 cm, h=2.5 mm) rolled up and fitted inside a syringe (d=0.9 cm); (II) a photograph of the hydrogel once injected into a saline solution, (III) a photograph of the hydrogel unfurled to its original shape.

The cardiac tissues' dimensions were chosen to accurately reflect the clinical requirements of a cardiac patch, both in terms of its own size and the size of the trocar through which it would be inserted [Gao et al, Circulation 2018, 137, 1712; Migliore and Deodato, Surg. Endosc. 2001, 15, 899].

Following the application of the stresses, the tissues were reassessed to ensure that the process was performed without causing damage.

The nanostructure of the injected tissues was imaged by SEM and the obtained images are shown in FIG. 5B (before injection) and 5C (after injection); Scale bar=400 nm. Despite the stresses endured by the tissue during injection, the fibrous network of the tissue's ECM proved to be sufficiently elastic, and there was no change in the ECM's nanostructure following injection. In contrast, printed cardiac tissues, without the addition of SOx, were physically torn during injection (See, FIG. 5A, IV).

As the hollow lumen forms a particularly weak spot in the tissue, tissues were re-perfused post-injection to ensure the continued integrity of the vascular network. As can be seen in FIG. 5D (Scale bar=3 mm), following injection, the blood vessels remained intact and perfusable.

Endothelial cells were stained and imaged to assess their morphology, and the images are presented in FIG. 5E (Scale bar=100 micrometer). As can be seen in the top panel, a top view of the cells shows a confluent layer of endothelial cells lining the blood vessel, indicating that in spite of the stresses applied, the cells remained confluent and formed a complete endothelial barrier. A side-view image created by confocal microscopy (bottom panel) shows the empty lumen surrounded by endothelial cells.

Cardiomyocyte morphology was also re-assessed and immunostaining image is presented in FIG. 5F (Scale bar=20 micrometer). The cells were shown to maintain their elongated morphology, indicative of the patch's ability to contract. The 3D arrangement of the cardiomyocytes is undisturbed by the process of injecting the tissue.

The tissue functionality was assessed after injection. Immediately post-injection, the cardiac tissue stopped beating. However, as can be seen in FIG. 5G, spontaneous contractions could be observed already after 30 minutes, and full recovery occurred within three hours, at which time, spontaneous contractions returned to their pre-injection levels and had fully recovered the baseline contraction rate and amplitude.

At this point, calcium transients were re-imaged to ensure that the tissues' contractions were still accompanied by smooth propagation of an action potential. The obtained data is shown in FIG. 5H, as follows: (I) distinct points, represented as colored circles, were selected from the overall image; (II) the fluorescent signal was tracked as a function of time at each point, and the derivative of the plot was taken as a function of time; (III) a single action potential was isolated and tracked as it moved across the indicated points, showing a clear progression of the signal; (IV) an individual pulse plotted as it moved across the tissue. The tissue continued to demonstrate healthy contractions and a high degree of interconnectedness and action potential transients following its injection.

This experiment revealed a similar contraction pattern as pre-injection, indicating that the reinforced tissue hydrogel was able to cushion the cells within and protect them from external forces.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A method for reinforcing an engineered cellularized construct fabricated from extracellular matrix (ECM) hydrogel and cells, the method comprising contacting the engineered cellularized construct with a biocompatible small-molecule reinforcing agent that is capable of chemically interacting with the ECM-based hydrogel under conditions that maintain viability of the cells, to thereby increase a compressive modulus of the ECM-based hydrogel by at least 10%, wherein said construct is devoid of retinal pigment epithelial (RPE) cells.

2. The method of claim 1, wherein said chemically interacting effects cross-linking of the ECM-based hydrogel.

3. The method of claim 1, wherein said reinforcing agent is capable of chemically interacting with the ECM-based hydrogel via a Click reaction.

4. The method of claim 1, wherein said reinforcing agent is a polyaldehyde.

5. The method of claim 1, wherein said reinforcing agent is an oxidized, poly-aldehyde saccharide.

6. The method of claim 1, wherein said cells comprise at least two different cell types.

7. The method of claim 1, wherein said contacting is effected following culturing the cells of said cellularized engineered construct for a length of time such that said at least a portion of said cells interact biologically with one another.

8. The method of claim 1, wherein said engineered cellularized construct is generated by sequentially forming a plurality of layers on a receiving medium in a configured pattern corresponding to the shape of the engineered construct by 3D bioprinting, wherein for at least a few of said layers said forming is effected by dispensing of at least one bioink composition that comprises said ECM-based hydrogel and said cells.

9. The method of claim 8, wherein said dispensing is of at least two bioink compositions, at least one of the bioink compositions comprises said ECM-based hydrogel and a first type of cells, and at least another one of the bioink compositions comprises a second type of cells which is different from said first type of cells.

10. The method of claim 1, further comprising perfusing said cellularized engineered construct.

11. A cellularized engineered construct obtainable by the method of claim 1.

12. A method of treating a condition associated with a damaged tissue in a subject in need thereof, the method comprising implanting the cellularized engineered construct of claim 11 in the subject, thereby treating the condition associated with the damaged tissue.

13. A method of preparing a cellularized engineered construct, the method comprising:
encapsulating cells in the presence of an ECM-based hydrogel, to thereby provide a bioink composition;
depositing said bioink composition in a configured pattern corresponding to the shape of the engineered construct;
culturing said cells of the engineered construct; and
subsequent to said culturing, contacting said cellularized engineered construct with a reinforcing agent, said reinforcing agent being a biocompatible small-molecule reinforcing agent that is capable of chemically interacting with the ECM-based hydrogel under conditions that maintain viability of the cells, to thereby increase a compressive modulus of the ECM-based hydrogel by at least 10%, wherein said construct is devoid of RPE cells.

14. The method of claim 13, wherein said chemically interacting effects cross-linking of the ECM-based hydrogel.

15. The method of claim 13, wherein said reinforcing agent is capable of chemically interacting with the ECM-based hydrogel via a Click reaction.

16. The method of claim 13, wherein said reinforcing agent is a polyaldehyde.

17. The method of claim 13, wherein said reinforcing agent is an oxidized, poly-aldehyde saccharide.

18. The method of claim 13, wherein said cells comprise at least two different cell types.

19. The method of claim 13, wherein said contacting is effected following culturing the cells of said cellularized engineered construct for a length of time such that said at least a portion of said cells interact biologically with one another.

20. The method of claim 13, further comprising perfusing said cellularized engineered construct.

21. The method of claim 20, wherein said perfusing is effected subsequent to contacting said cellularized engineered construct with said reinforcing agent.

22. A cellularized engineered construct obtainable by the method of claim 13.

23. A method of treating a condition associated with a damaged tissue in a subject in need thereof, the method comprising implanting the cellularized engineered construct of claim 22 in the subject, thereby treating the condition associated with the damaged tissue.

24. A cellularized engineered construct comprising cells distributed within a chemically cross-linked ECM-based hydrogel, wherein said ECM-based hydrogel is chemically cross-linked by a biocompatible small-molecule reinforcing agent that is capable of chemically interacting with the ECM-based hydrogel under conditions that maintain viability of the cells, and wherein a compressive modulus of the ECM-based hydrogel is higher by at least 10% than a compressive modulus of the ECM-based hydrogel which is not chemically cross-linked, wherein said construct is devoid of RPE cells.

25. The cellularized engineered construct of claim 24, wherein said biocompatible small-molecule reinforcing agent is chemically interacted with at least 10% of chemically compatible groups present in the ECM-based hydrogel before chemically interacting with said reinforcing agent, said chemically compatible groups are those that are capable of chemically interacting with the reinforcing agent under conditions that maintain viability of the cells.

26. The cellularized engineered construct of claim 24, wherein said chemically interacting effects cross-linking of the ECM-based hydrogel.

27. The cellularized engineered construct of claim 24, wherein said reinforcing agent is capable of chemically interacting with the ECM-based hydrogel via a Click reaction.

28. The cellularized engineered construct of claim 24, wherein said reinforcing agent is a polyaldehyde.

29. The cellularized engineered construct of claim 24, wherein said reinforcing agent is an oxidized, poly-aldehyde saccharide.

30. The cellularized engineered construct of claim 24, wherein said cells comprises at least two different cell types.

31. A method of treating a condition associated with a damaged tissue in a subject in need thereof, the method comprising implanting the cellularized engineered construct of claim 24 in the subject, thereby treating the condition associated with the damaged tissue.
